SEQUENCE LISTING (42 sequences)

SEQ ID NO: 1 - DNA, 984 bp, Mus sp.
  source 1..984: mol_type = genomic DNA
atgtgccaccagcagctggtgatcagctggttcagcctggtgttcctggccagccccctggtggccatctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgaggtgcgaggccaagaactacagcggcaggttcacctgctggtggctgaccaccatcagcaccgacctgaccttcagcgtgaagagcagcaggggcagcagcgacccccagggcgtgacctgcggcgccgccaccctgagcgccgagagggtgaggggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatcagggacatcatcaagcccgacccccccaagaacctgcagctgaagcccctgaagaacagcaggcaggtggaggtgagctgggagtaccccgacacctggagcaccccccacagctacttcagcctgaccttctgcgtgcaggtgcagggcaagagcaagagggagaagaaggacagggtgttcaccgacaagaccagcgccaccgtgatctgcaggaagaacgccagcatcagcgtgagggcccaggacaggtactacagcagcagctggagcgagtgggccagcgtgccctgcagc

SEQ ID NO: 2 - DNA, 429 bp, Mus sp.
  source 1..429: mol_type = genomic DNA
atggagcccgccggccccgcccccggcaggctgggccccctgctgctgtgcctgctgctgagcgccagctgcttctgcaccggcgccaccggcaaggagctgaaggtgacccagcccgagaagagcgtgagcgtggccgccggcgacagcaccgtgctgaactgcaccctgaccagcctgctgcccgtgggccccatcaggtggtacaggggcgtgggccccagcaggctgctgatctacagcttcgccggcgagtacgtgcccaggatcaggaacgtgagcgacaccaccaagaggaacaacatggacttcagcatcaggatcagcaacgtgacccccgccgacgccggcatctactactgcgtgaagttccagaagggcagcagcgagcccgacaccgagatccagagcggcggcggcaccgaggtg

SEQ ID NO: 3 - DNA, 90 bp, Mus sp.
  source 1..90: mol_type = genomic DNA
atggagcccgccggccccgcccccggcaggctgggccccctgctgctgtgcctgctgctgagcgccagctgcttctgcaccggcgccacc

SEQ ID NO: 4 - DNA, 321 bp, Mus sp.
  source 1..321: mol_type = genomic DNA
ggcaaggagctgaaggtgacccagcccgagaagagcgtgagcgtggccgccggcgacagcaccgtgctgaactgcaccctgaccagcctgctgcccgtgggccccatcaggtggtacaggggcgtgggccccagcaggctgctgatctacagcttcgccggcgagtacgtgcccaggatcaggaacgtgagcgacaccaccaagaggaacaacatggacttcagcatcaggatcagcaacgtgacccccgccgacgccggcatctactactgcgtgaagttccagaagggcagcagcgagcccgacaccgagatccagagc

SEQ ID NO: 5 - DNA, 1512 bp, Homo sapiens
  source 1..1512: mol_type = genomic DNA
atggagcccgccggccccgcccccggcaggctgggccccctgctgtgcctgctgctggccgccagctgcgcctggagcggcgtggccggcgaggaggagctgcaggtgatccagcccgacaagagcgtgctggtggccgccggcgagaccgccaccctgaggtgcaccgccaccagcctgatccccgtgggccccatccagtggttcaggggcgccggccccggcagggagctgatctacaaccagaaggagggccacttccccagggtgaccaccgtgagcgacctgaccaagaggaacaacatggacttcagcatcaggatcggcaacatcacccccgccgacgccggcacctactactgcgtgaagttcaggaagggcagccccgacgacgtggagttcaagagcggcgccggcaccgagctgagcgtgagggccaagcccagcgcccccgtggtgagcggccccgccgccagggccaccccccagcacaccgtgagcttcacctgcgagagccacggcttcagccccagggacatcaccctgaagtggttcaagaacggcaacgagctgagcgacttccagaccaacgtggaccccgtgggcgagagcgtgagctacagcatccacagcaccgccaaggtggtgctgaccagggaggacgtgcacagccaggtgatctgcgaggtggcccacgtgaccctgcagggcgaccccctgaggggcaccgccaacctgagcgagaccatcagggtgccccccaccctggaggtgacccagcagcccgtgagggccgagaaccaggtgaacgtgacctgccaggtgaggaagttctacccccagaggctgcagctgacctggctggagaacggcaacgtgagcaggaccgagaccgccagcaccgtgaccgagaacaaggacggcacctacaactggatgagctggctgctggtgaacgtgagcgcccacagggacgacgtgaagctgacctgccaggtggagcacgacggccagcccgccgtgagcaagagccacgacctgaaggtgagcgcccaccccaaggagcagggcagcaacaccgccgccgagaacaccggcagcaacgagaggaacatctacatcgtggtgggcgtggtgtgcaccctgctggtggccctgctgatggccgccctgtacctggtgaggatcaggcagaagaaggcccagggcagcaccagcagcaccaggctgcacgagcccgagaagaacgccagggagatcacccaggacaccaacgacatcacctacgccgacctgaacctgcccaagggcaagaagcccgccccccaggccgccgagcccaacaaccacaccgagtacgccagcatccagaccagcccccagcccgccagcgaggacaccctgacctacgccgacctggacatggtgcacctgaacaggacccccaagcagcccgcccccaagcccgagcccagcttcagcgagtacgccagcgtgcaggtgcccaggaag

SEQ ID NO: 6 - DNA, 429 bp, Homo sapiens
  source 1..429: mol_type = genomic DNA
atggagcccgccggccccgcccccggcaggctgggccccctgctgtgcctgctgctggccgccagctgcgcctggagcggcgtggccggcgaggaggagctgcaggtgatccagcccgacaagagcgtgctggtggccgccggcgagaccgccaccctgaggtgcaccgccaccagcctgatccccgtgggccccatccagtggttcaggggcgccggccccggcagggagctgatctacaaccagaaggagggccacttccccagggtgaccaccgtgagcgacctgaccaagaggaacaacatggacttcagcatcaggatcggcaacatcacccccgccgacgccggcacctactactgcgtgaagttcaggaagggcagccccgacgacgtggagttcaagagcggcgccggcaccgagctgagc

SEQ ID NO: 7 - DNA, 90 bp, Homo sapiens
  source 1..90: mol_type = genomic DNA
atggagcccgccggccccgcccccggcaggctgggccccctgctgtgcctgctgctggccgccagctgcgcctggagcggcgtggccggc

SEQ ID NO: 8 - DNA, 318 bp, Homo sapiens
  source 1..318: mol_type = genomic DNA
gaggagctgcaggtgatccagcccgacaagagcgtgctggtggccgccggcgagaccgccaccctgaggtgcaccgccaccagcctgatccccgtgggccccatccagtggttcaggggcgccggccccggcagggagctgatctacaaccagaaggagggccacttccccagggtgaccaccgtgagcgacctgaccaagaggaacaacatggacttcagcatcaggatcggcaacatcacccccgccgacgccggcacctactactgcgtgaagttcaggaagggcagccccgacgacgtggagttcaagagcggc

SEQ ID NO: 9 - DNA, 351 bp, Mus sp.
  source 1..351: mol_type = genomic DNA
gccaccaggaccgaggtgaaggtgatccagcccgagaagagcgtgagcgtggccgccggcgacagcaccgtgctgaactgcaccctgaccagcctgctgcccgtgggccccatcaggtggtacaggggcgtgggccagagcaggcagctgatctacagcttcaccaccgagcacttccccagggtgaccaacgtgagcgacgccaccaagaggagcaacctggacttcagcatcaggatcagcaacgtgacccccgaggacgccggcacctactactgcgtgaagttccagaggggcagccccgacaccgagatccagagcggcggcggcaccgaggtgtacgtgctggcc

SEQ ID NO: 10 - protein, 513 aa, Mus sp.
MEPAGPAPGRLGPLLLCLLLSASCFCTGATGKELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEVYVLAKPSPPEVSGPADRGIPDQKVNFTCKSHGFSPRNITLKWFKDGQELHPLETTVNPSGKNVSYNISSTVRVVLNSMDVNSKVICEVAHITLDRSPLRGIANLSNFIRVSPTVKVTQQSPTSMNQVNLTCRAERFYPEDLQLIWLENGNVSRNDTPKNLTKNTDGTYNYTSLFLVNSSAHREDVVFTCQVKHDQQPAITRNHTVLGFAHSSDQGSMQTFPDNNATHNWNVFIGVGVACALLVVLLMAALYLLRIKQKKAKGSTSSTRLHEPEKNAREITQVQSLIQDTNDINDITYADLNLPKEKKPAPRAPEPNNHTEYASIETGKVPRPEDTLTYADLDMVHLSRAQPAPKPEPSFSEYASVQVQRK

SEQ ID NO: 11 - protein, 143 aa, Mus sp.
MEPAGPAPGRLGPLLLCLLLSASCFCTGATGKELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEV

SEQ ID NO: 12 - protein, 31 aa, Mus sp.
MEPAGPAPGRLGPLLLCLLLSASCFCTGATG

SEQ ID NO: 13 - protein, 106 aa, Mus sp.
KELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQS

SEQ ID NO: 14 - protein, 504 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 15 - protein, 143 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELS

SEQ ID NO: 16 - protein, 30 aa, Homo sapiens
MEPAGPAPGRLGPLLCLLLAASCAWSGVAG

SEQ ID NO: 17 - protein, 106 aa, Homo sapiens
EELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSG

SEQ ID NO: 18 - protein, 146 aa, Mus sp.
MEPAGPAPGRLGPLLCLLLAASCAWSGVAGETEVKVIQPEKSVSVAAGDSTVLNCTLTSLLPVGPIRWYRGVGQSRQLIYSFTTEHFPRVTNVSDATKRSNLDFSIRISNVTPEDAGTYYCVKFQRGSPDTEIQSGGGTEVYVLAK

SEQ ID NO: 19 - protein, 436 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 347 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 444 aa, synthetic construct
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 219 aa, synthetic construct
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 130 bp, synthetic construct
  source 1..130: mol_type = other DNA
tcactaattccaaacccacccgctttttatagtaagtttttcacccataaataataaatacaataattaatttctcgtaaaagtagaaaatatattctaatttattgcacggtaaggaagtagatcataa

SEQ ID NO: 24 - DNA, 34 bp, synthetic construct
  source 1..34: mol_type = other DNA
aaaaattgaaaaactagcgtctttttttgctcga

SEQ ID NO: 25 - DNA, 37 bp, synthetic construct
  source 1..37: mol_type = other DNA
aagctcacaaaaaaaacttctctaaatagattcgagc

SEQ ID NO: 26 - DNA, 64 bp, synthetic construct
  source 1..64: mol_type = other DNA
ggatcataaaaatatagtagaatttcattttgtttttttctatgctataaatagcttctcgagc

SEQ ID NO: 27 - DNA, 37 bp, synthetic construct
  source 1..37: mol_type = other DNA
cctgcagggcgtcctaaaattaaaacaaatgatataa

SEQ ID NO: 28 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 29 - DNA, 511 bp, synthetic construct
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 30 - DNA, 1000 bp, synthetic construct
  source 1..1000: mol_type = other DNA
atttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattgattactataaatggagaatgttcctaatgtatactttaatcctgtgtttatagagcccacgtttaaacattctttattaagtgtttataaacacagattaatagttttatttgaagtattcgttgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatc

SEQ ID NO: 31 - DNA, 34 bp, synthetic construct
  source 1..34: mol_type = other DNA
ataacttcgtatagcatacattatacgaagttat

SEQ ID NO: 32 - DNA, 12 bp, synthetic construct
  source 1..12: mol_type = other DNA
aattctgcagat

SEQ ID NO: 33 - DNA, 717 bp, synthetic construct
  source 1..717: mol_type = other DNA
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtcccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaataa

SEQ ID NO: 34 - DNA, 12 bp, synthetic construct
  source 1..12: mol_type = other DNA
aaggccgctcga

SEQ ID NO: 35 - DNA, 997 bp, synthetic construct
  source 1..997: mol_type = other DNA
tattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaaagtagttgttaactttgattatcctaatcaggaatatgactattttcacatgtactttatgctgaggaccgtgtactgcaataaaacatttcctactactaaagcaaaggtactatttctacaacaatctatatttcgtttcttaaatattccaacaacatcaactgaaaaagttagtcatgaaccaatacaacg

SEQ ID NO: 36 - DNA, 2610 bp, synthetic construct
  source 1..2610: mol_type = other DNA
atttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattgattactataaatggagaatgttcctaatgtatactttaatcctgtgtttatagagcccacgtttaaacattctttattaagtgtttataaacacagattaatagttttatttgaagtattcgttgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatccctgcaggtcactaattccaaacccacccgctttttatagtaagtttttcacccataaataataaatacaataattaatttctcgtaaaagtagaaaatatattctaatttattgcacggtaaggaagtagatcataaactagtatggagcccgccggccccgcccccggcaggctgggccccctgctgctgtgcctgctgctgagcgccagctgcttctgcaccggcgccaccggcaaggagctgaaggtgacccagcccgagaagagcgtgagcgtggccgccggcgacagcaccgtgctgaactgcaccctgaccagcctgctgcccgtgggccccatcaggtggtacaggggcgtgggccccagcaggctgctgatctacagcttcgccggcgagtacgtgcccaggatcaggaacgtgagcgacaccaccaagaggaacaacatggacttcagcatcaggatcagcaacgtgacccccgccgacgccggcatctactactgcgtgaagttccagaagggcagcagcgagcccgacaccgagatccagagcggcggcggcaccgaggtggagctcataacttcgtatagcatacattatacgaagttattattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaaagtagttgttaactttgattatcctaatcaggaatatgactattttcacatgtactttatgctgaggaccgtgtactgcaataaaacatttcctactactaaagcaaaggtactatttctacaacaatctatatttcgtttcttaaatattccaacaacatcaactgaaaaagttagtcatgaaccaatacaacg

SEQ ID NO: 37 - DNA, 3987 bp, synthetic construct
  source 1..3987: mol_type = other DNA
atttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattgattactataaatggagaatgttcctaatgtatactttaatcctgtgtttatagagcccacgtttaaacattctttattaagtgtttataaacacagattaatagttttatttgaagtattcgttgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatccctgcaggtcactaattccaaacccacccgctttttatagtaagtttttcacccataaataataaatacaataattaatttctcgtaaaagtagaaaatatattctaatttattgcacggtaaggaagtagatcataaactagtatgtgccaccagcagctggtgatcagctggttcagcctggtgttcctggccagccccctggtggccatctgggagctgaagaaggacgtgtacgtggtggagctggactggtaccccgacgcccccggcgagatggtggtgctgacctgcgacacccccgaggaggacggcatcacctggaccctggaccagagcagcgaggtgctgggcagcggcaagaccctgaccatccaggtgaaggagttcggcgacgccggccagtacacctgccacaagggcggcgaggtgctgagccacagcctgctgctgctgcacaagaaggaggacggcatctggagcaccgacatcctgaaggaccagaaggagcccaagaacaagaccttcctgaggtgcgaggccaagaactacagcggcaggttcacctgctggtggctgaccaccatcagcaccgacctgaccttcagcgtgaagagcagcaggggcagcagcgacccccagggcgtgacctgcggcgccgccaccctgagcgccgagagggtgaggggcgacaacaaggagtacgagtacagcgtggagtgccaggaggacagcgcctgccccgccgccgaggagagcctgcccatcgaggtgatggtggacgccgtgcacaagctgaagtacgagaactacaccagcagcttcttcatcagggacatcatcaagcccgacccccccaagaacctgcagctgaagcccctgaagaacagcaggcaggtggaggtgagctgggagtaccccgacacctggagcaccccccacagctacttcagcctgaccttctgcgtgcaggtgcagggcaagagcaagagggagaagaaggacagggtgttcaccgacaagaccagcgccaccgtgatctgcaggaagaacgccagcatcagcgtgagggcccaggacaggtactacagcagcagctggagcgagtgggccagcgtgccctgcagcggcggcggcggcagcggcggcggcggcagcggcggcggcggcagcggcggcggcggcagcgccagctggccccccggcagcgccagccagcccccccccagccccgccgccgccaccggcctgcaccccgccgccaggcccgtgagcctgcagtgcaggctgagcatgtgccccgccaggagcctgctgctggtggccaccctggtgctgctggaccacctgagcctggccaggaacctgcccgtggccacccccgaccccggcatgttcccctgcctgcaccacagccagaacctgctgagggccgtgagcaacatgctgcagaaggccaggcagaccctggagttctacccctgcaccagcgaggagatcgaccacgaggacatcaccaaggacaagaccagcaccgtggaggcctgcctgcccctggagctgaccaagaacgagagctgcctgaacagcagggagaccagcttcatcaccaacggcagctgcctggccagcaggaagaccagcttcatgatggccctgtgcctgagcagcatctacgaggacctgaagatgtaccaggtggagttcaagaccatgaacgccaagctgctgatggaccccaagaggcagatcttcctggaccagaacatgctggccgtgatcgacgagctgatgcaggccctgaacttcaacagcgagaccgtgccccagaagagcagcctggaggagcccgacttctacaagaccaagatcaagctgtgcatcctgctgcacgccttcaggatcagggccgtgaccatcgacagggtgatgagctacctgaacgccagcgagctcataacttcgtatagcatacattatacgaagttattattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaaagtagttgttaactttgattatcctaatcaggaatatgactattttcacatgtactttatgctgaggaccgtgtactgcaataaaacatttcctactactaaagcaaaggtactatttctacaacaatctatatttcgtttcttaaatattccaacaacatcaactgaaaaagttagtcatgaaccaatacaacg

SEQ ID NO: 38 - DNA, 1000 bp, synthetic construct
  source 1..1000: mol_type = other DNA
gtacgtactatgccaaatgaatcacgtgttaaacaatgggtgatggatacacttaatggtataatgatgaatgaacgcgatgtttctgtaagcgttggcaccggaatactattcatggaaatgtttttcgattacaataaaaatagtatcaacaatcaactaatgtatgatataattaatagcgtatctataattctagctaatgagagatatagaagcgcttttaacgacgatggtatatacatccgtagaaatatgattaacaagttgtacggatacgcatctctaactactattggcacgatcgctggaggtgtttgttattatctgttgatgcatctagttagtttgtataaataattatttcaatatactagttaaaattttaagattttaaatgtataaaaaactaataacgtttttatttgtaataggtgcattagcatcctattcgaataatgagtacactccgtttaataaactgagtgtaaaactctatatagatggagtagataatatagaaaattcatatactgatgataataatgaattggtgttaaattttaaagagtacacaatttctattattacagagtcatgcgacgtcggatttgattccatagatatagatgttataaacgactataaaattattgatatgtataccattgactcgtctactattcaacgcagaggtcacacgtgtagaatatctaccaaattatcatgccattatgataagtacccttatattcacaaatatgatggtgatgagcgacaatattctattactgcagagggaaaatgctataaaggaataaaatatgaaataagtatgatcaacgatgatactctattgagaaaacatactcttaaaattggatctacttatatatttgatcgtcatggacatagtaatacatattattcaaaatatgatttttaaaaatttaaaatatattatcacttcagtgacagtagtcaaataacaaacaacacc

SEQ ID NO: 39 - DNA, 20 bp, synthetic construct
  source 1..20: mol_type = other DNA
cgaagttataattctgcaga

SEQ ID NO: 40 - DNA, 717 bp, synthetic construct
  source 1..717: mol_type = other DNA
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtcccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatag

SEQ ID NO: 41 - DNA, 867 bp, synthetic construct
  source 1..867: mol_type = other DNA
ctaatttttattaatgatacaaaaacgaaataaaactgcatattatacactggttaacgcccttataggctctaaccattttcaagatgaggtccctgattatagtccttctgttcccctctatcatctactccatgtctattagacaatgtgagaagactgaagaggaaacatggggattgaaaatagggttgtgtataattgccaaagatttctatcccgaaagaactgattgcagtgttcatctcccaactgcaagtgaaggattgataactgaaggcaatggattcagggatatacgaaacaccgataaattataaaaaaagcaatgtgtccgctgtttccgttaataatactattttcgtaactggcggattattcataaataactctaatagcacgatcgtggttaacaatatggaaaaacttgacatttataaagacaaacaatggtcgattatagaaatgcctatggctagggtatatcacggcatcgactcgacatttggaatgttatattttgccggaggtctatccgttaccgaacaatatggtaatttagagaaaaacaacgagatatcttgttacaatcctagaacgaataagtggtttgatatttcatatactatttataagatatccatatcatcattgtgtaaactaaataacgtcttctatgtatttagtaaggacattggatatgtggaaaagtatgatggtgcatggaagttagtacatgatcgtctccccgctataaaggcattatcaacttctccttattgattgaaaatgaaaatataaatagtttttatgtatagcagtattaccctatagttttattgcttactactaacatggatacagatg

SEQ ID NO: 42 - DNA, 1867 bp, synthetic construct
  source 1..1867: mol_type = other DNA
gtacgtactatgccaaatgaatcacgtgttaaacaatgggtgatggatacacttaatggtataatgatgaatgaacgcgatgtttctgtaagcgttggcaccggaatactattcatggaaatgtttttcgattacaataaaaatagtatcaacaatcaactaatgtatgatataattaatagcgtatctataattctagctaatgagagatatagaagcgcttttaacgacgatggtatatacatccgtagaaatatgattaacaagttgtacggatacgcatctctaactactattggcacgatcgctggaggtgtttgttattatctgttgatgcatctagttagtttgtataaataattatttcaatatactagttaaaattttaagattttaaatgtataaaaaactaataacgtttttatttgtaataggtgcattagcatcctattcgaataatgagtacactccgtttaataaactgagtgtaaaactctatatagatggagtagataatatagaaaattcatatactgatgataataatgaattggtgttaaattttaaagagtacacaatttctattattacagagtcatgcgacgtcggatttgattccatagatatagatgttataaacgactataaaattattgatatgtataccattgactcgtctactattcaacgcagaggtcacacgtgtagaatatctaccaaattatcatgccattatgataagtacccttatattcacaaatatgatggtgatgagcgacaatattctattactgcagagggaaaatgctataaaggaataaaatatgaaataagtatgatcaacgatgatactctattgagaaaacatactcttaaaattggatctacttatatatttgatcgtcatggacatagtaatacatattattcaaaatatgatttttaaaaatttaaaatatattatcacttcagtgacagtagtcaaataacaaacaacaccctaatttttattaatgatacaaaaacgaaataaaactgcatattatacactggttaacgcccttataggctctaaccattttcaagatgaggtccctgattatagtccttctgttcccctctatcatctactccatgtctattagacaatgtgagaagactgaagaggaaacatggggattgaaaatagggttgtgtataattgccaaagatttctatcccgaaagaactgattgcagtgttcatctcccaactgcaagtgaaggattgataactgaaggcaatggattcagggatatacgaaacaccgataaattataaaaaaagcaatgtgtccgctgtttccgttaataatactattttcgtaactggcggattattcataaataactctaatagcacgatcgtggttaacaatatggaaaaacttgacatttataaagacaaacaatggtcgattatagaaatgcctatggctagggtatatcacggcatcgactcgacatttggaatgttatattttgccggaggtctatccgttaccgaacaatatggtaatttagagaaaaacaacgagatatcttgttacaatcctagaacgaataagtggtttgatatttcatatactatttataagatatccatatcatcattgtgtaaactaaataacgtcttctatgtatttagtaaggacattggatatgtggaaaagtatgatggtgcatggaagttagtacatgatcgtctccccgctataaaggcattatcaacttctccttattgattgaaaatgaaaatataaatagtttttatgtatagcagtattaccctatagttttattgcttactactaacatggatacagatg

(19) United States
(12) Patent Application Publication
(10) Pub. No.: US 2025/0302899 A1
(43) Pub. Date: Oct. 2, 2025

(54) COMPOSITIONS OF CD47/SIRP-ALPHA IMMUNE CHECKPOINT INHIBITORS AND USES THEREOF

(71) Applicant: KaliVir Immunotherapeutics, Inc., Pittsburgh, PA (US)

(72) Inventors: Stephen Howard THORNE, Pittsburgh, PA (US); Ravikumar MUTHUSWAMY, Pittsburgh, PA (US); Mingrui ZHANG, Pittsburgh, PA (US)

(21) Appl. No.: 19/237,404

(22) Filed: Jun. 13, 2025

Related U.S. Application Data
(63) Continuation of application No. PCT/US2023/084001, filed on Dec. 14, 2023.
(60) Provisional application No. 63/432,789, filed on Dec. 15, 2022.

(51) Int. Cl. A61K 35/768; C07K 14/705; C07K 16/28; C12N 7/00

(52) U.S. Cl. CPC A61K 35/768; C07K 14/70503; C07K 16/2896; C12N 7/00; C07K 2319/00; C12N 2710/24122; C12N 2710/24132

(57) ABSTRACT

The present disclosure provides for an oncolytic virus comprising an exogenous nucleic acid encoding for a polypeptide that acts as a CD47-SIRP-alpha immune checkpoint inhibitor. Oncolytic viruses optionally comprise a mutation or deletion of the gene expressing IFN-gamma. Compositions described herein are further described for use in the treatment of cancer.

### CROSS-REFERENCE

This application claims the benefit of PCT/US2023/084001 filed Dec. 14, 2023, which claims the benefit of U.S. Provisional Application No. 63/432,789 filed Dec. 15, 2022, both of which are incorporated by reference herein in their entirety.

### SEQUENCE LISTING

The instant application contains a Sequence Listing which has been submitted electronically in XML format and is hereby incorporated by reference in its entirety. Said XML copy, created on Nov. 7, 2023, is named 199249-724601_SL.xml and is 59,345 bytes in size.

### BACKGROUND

Cancer remains a major source of illness globally. Biologics delivery tools for use in treatment of cancer face challenges around specificity to target locations and local expression stability. In addition, tumor cells employ various mechanisms to avoid detection attack by the host immune system. Such mechanisms can influence the effectiveness of cancer immunotherapies. Thus, there is a need for improved therapies for targeted biologic delivery for treatment of cancer that also counter immune system evasion by tumor cells.

### BRIEF SUMMARY

Described herein are compositions, wherein the composition comprises: an oncolytic virus comprising an exogenous nucleic acid comprising a sequence encoding for a soluble SIRP-alpha polypeptide or functional fragment thereof.

Described herein are compositions, wherein the composition comprises: an oncolytic virus comprising an exogenous nucleic acid comprising a sequence encoding for an anti-CD47 antibody or fragment thereof.

Described herein are compositions, wherein the composition comprises: an oncolytic virus, wherein the oncolytic virus comprises: an insertion at a TK gene locus comprising, in 5′ to 3′ order: a promoter region, wherein the promoter is P7.5; and a region encoding a soluble SIRP-alpha polypeptide or functional fragment thereof.

Described herein are compositions, wherein the composition comprises: an oncolytic virus, wherein the oncolytic virus comprises: an insertion at a TK gene locus comprising, in 5′ to 3′ order: a promoter region, wherein the promoter is P10; a region encoding a soluble SIRP-alpha polypeptide or functional fragment thereof.

Described herein are compositions, wherein the composition comprises: an oncolytic virus, wherein the oncolytic virus comprises: an insertion at a TK gene locus comprising, in 5′ to 3′ order: a promoter region, wherein the promoter is P7.5; and a region encoding an anti-CD47 antibody or fragment thereof.

Described herein are compositions, wherein the composition comprises: an oncolytic virus, wherein the oncolytic virus comprises: an insertion at a TK gene locus comprising, in 5′ to 3′ order: a promoter region, wherein the promoter is P10; a region encoding an anti-CD47 antibody or fragment thereof.

Described herein are pharmaceutical compositions, wherein the pharmaceutical composition comprises: a composition as described herein; and a pharmaceutically acceptable excipient.

Described herein are methods for treatment of cancer comprising administering to a subject having cancer a pharmaceutical composition as described herein in an amount sufficient for treatment of a cancer.

Described herein are methods for activating an anti-tumor immune response, comprising administering to a subject having a cancer a pharmaceutical composition as described herein.

Described herein are methods for reduction of incidence of tumor cell growth, comprising: administering to tumor cells a pharmaceutical composition as described herein in an effective amount sufficient for reduction of incidence of tumor cell growth.

### BRIEF DESCRIPTION OF THE DRAWINGS

The novel features of the disclosure are set forth with particularity in the appended claims. A better understanding of the features and advantages of this disclosure will be obtained by reference to the following detailed description that sets forth illustrative embodiments, in which the principles of this disclosure are utilized, and the accompanying drawings of which:

FIG. 1A is a schematic representation of the CD47/SIRP-alpha interaction between a tumor cell and a macrophage.

FIG. 1B is a schematic representation of a domain of SIRP-alpha preventing the CD47/SIRP-alpha interaction.

FIG. 2 is a diagram of a transgene inserted in the TK locus with a P7.5 promoter driving expression of a sequence encoding a CD47 binding domain of SIRP-alpha.

### DETAILED DESCRIPTION

Tumor cells employ various mechanisms to avoid detection attack by the host immune system. Such mechanisms can influence the effectiveness of cancer immunotherapies. Described herein are compositions comprising a combination of immune checkpoint inhibitor and pro-inflammatory cytokine, in order to enhance an immune response to tumor cells, either alone or in conjunction with other therapeutic modalities.

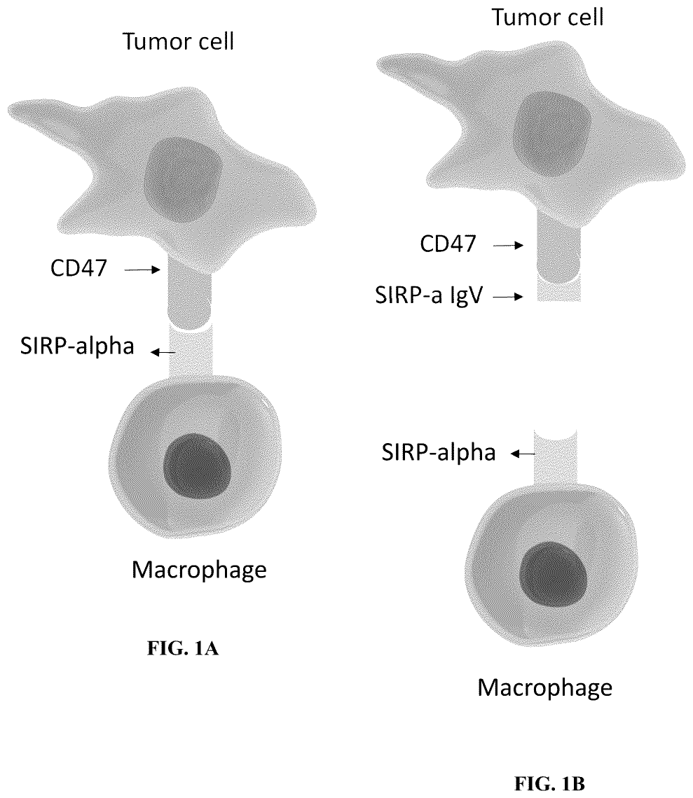

Signal regulatory protein alpha (SIRP-alpha) is a regulatory membrane glycoprotein expressed by at least macrophages, myeloid cells, stem cells, and neurons. SIRP-alpha acts as a checkpoint to cellular immune response by binding with CD47 and inhibiting effector functions of innate immune cells, such as phagocytosis by macrophages. When bound to CD47 expressed on a cell surface, the phagocytic response is suppressed. Cancer cells express CD47 to suppress activation of phagocytic or antigen presenting cells (APC), such as macrophages (MP) or dendritic cells (DC) as a form of immune-surveillance evasion (FIG. 1A). Binding of CD47 on tumor cells with SIRP-alpha downregulates a response and suppresses anti-tumor activity. Inhibition of the CD47/SIRP-alpha interaction, or immune checkpoint inhibition, can enhance macrophage response and increase anti-tumor activity. Described herein are oncolytic viruses comprising an exogenous nucleic acid encoding for a polypeptide that disrupts the CD47 SIRP-alpha interaction. In some embodiments, the polypeptide comprises a SIRP-alpha peptide. The domain of SIRP-alpha can bind CD47, acting as an inhibitor to the CD47-SIRP-alpha immune checkpoint (FIG. 1B), thereby allowing the macrophage response to proceed.

Provided herein are oncolytic viruses and uses thereof for treatment of cancer. Oncolytic viruses described herein can comprise one or more nucleic acids encoding for polypeptides as described herein. Nucleic acids provided herein can comprise DNA, RNA, nucleic acid analogues, or any combination thereof. Briefly, described herein are (1) nucleic acids encoding for a CD47-SIRP-alpha immune checkpoint inhibitor, (2) oncolytic viruses for expression of described inhibitors, (3) conditions for treatment, and (4) dosage amounts, forms, and methods of administration of compositions described herein.

### Definitions

The terminology used herein is for the purpose of describing particular cases only and is not intended to be limiting. As used herein, the singular forms “a”, “an” and “the” include the plural forms as well, unless the context clearly indicates otherwise. Furthermore, to the extent that the terms “contains,” “containing,” “including”, “includes,” “having,” “has”, “with”, or variants thereof are used in either the detailed description and/or the claims, such terms are intended to be inclusive in a manner similar to the term “comprising.”

The term “about” or “approximately” can mean within an acceptable error range for the particular value as determined by one of ordinary skill in the art, which will depend in part on how the value is measured or determined, e.g., the limitations of the measurement system. Where particular values are described in the application and claims, unless otherwise stated the term “about” should be assumed to mean an acceptable error range for the particular value, such as ±10% of the value modified by the term “about”.

The terms “heterologous nucleic acid sequence,” or “exogenous nucleic acid sequence,” or “transgenes,” as used herein, in relation to a specific virus can refer to a nucleic acid sequence that originates from a source other than the specified virus.

The term “mutation,” as used herein, can refer to a deletion, an insertion of a heterologous nucleic acid, an inversion, or a substitution, including an open reading frame ablating mutations as commonly understood in the art.

The term “gene,” as used herein, can refer to a segment of nucleic acid that encodes for an individual protein or RNA (also referred to as a “coding sequence” or “coding region”), optionally together with associated regulatory regions such as promoters, operators, terminators, and the like, which may be located upstream or downstream of the coding sequence.

A “promoter,” as used herein, can be a control sequence that is a region of a nucleic acid sequence at which initiation and rate of transcription are controlled. In certain embodiments, a promoter may contain genetic elements at which regulatory proteins and molecules may bind such as RNA polymerase and other transcription factors. The terms “operatively positioned,” “operatively linked,” “under control” and “under transcriptional control” can mean that a promoter is in a correct functional location and/or orientation in relation to a nucleic acid sequence to control transcriptional initiation and/or expression of that sequence. In certain embodiments, a promoter may or may not be used in conjunction with an “enhancer,” which refers to a cis-acting regulatory sequence involved in the transcriptional activation of a nucleic acid sequence.

The term “homology,” as used herein, may be to calculations of “homology” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=# of identical positions/total # of positions×100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The homology can be between the entire lengths of two sequences or between fractions of the entire lengths of two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA.

The term “subject” can refer to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” can be meant to include alleviating or abrogating a disorder, disease, or condition; or one or more of the symptoms associated with the disorder, disease, or condition; or alleviating or eradicating the cause(s) of the disorder, disease, or condition itself.

The term “therapeutically effective amount” can refer to the amount of a compound that, when administered, can be sufficient to prevent development of, or alleviate to some extent, one or more of the symptoms of the disorder, disease, or condition being treated.

The term “oncolytic,” as used herein, can refer to killing of cancer or tumor cells by an agent, such as an oncolytic poxvirus, such as an oncolytic vaccinia virus, e.g., through the direct lysis of said cells, by stimulating immune response towards said cells, apoptosis, expression of toxic proteins, autophagy and shutdown of protein synthesis, induction of anti-tumoral immunity, or any combinations thereof. The direct lysis of the cancer or tumor cells infected by the agent, such as an oncolytic vaccinia virus, can be a result of replication of the virus within said cells. In certain examples, the term “oncolytic,” can refer to killing of cancer or tumor cells without lysis of said cells.

The term “oncolytic virus” as used herein can refer to a virus that preferentially infects and kills tumor cells. In some embodiments, the oncolytic viruses can include, but are not limited to, (i) viruses that naturally replicate preferentially in cancer cells and are non-pathogenic in humans often due to elevated sensitivity to innate antiviral signaling or dependence on oncogenic signaling pathways; and (ii) viruses that are genetically-manipulated for use. In some embodiments, the oncolytic virus can be a measles virus, a poliovirus, a poxvirus, a vaccinia virus, an adenovirus, an adeno associated virus, a herpes simplex virus, a vesicular stomatitis virus, a reovirus, a Newcastle disease virus, a senecavirus, a lentivirus, a mengovirus, or a myxoma virus. In certain embodiments, the oncolytic virus can be a poxvirus. In certain embodiments, the oncolytic virus can be a vaccinia virus.

The term “modified oncolytic virus” as used herein can refer to an oncolytic virus that comprises a modification to its constituent, such as, but not limited to, a modification in the native genome (“backbone”) of the virus like a mutation or a deletion of a viral gene, introduction of an exogenous nucleic acid, a chemical modification of a viral nucleic acid or a viral protein, and introduction of an exogenous protein or modified viral protein to the viral capsid. In general, oncolytic viruses may be modified (also known as “engineered”) in order to gain improved therapeutic effects against tumor cells. In some embodiments, the oncolytic virus can be a modified poxvirus. In some embodiments, the oncolytic virus can be a modified poxvirus. In some embodiments, the oncolytic virus can be a modified vaccinia virus.

The terms “systemic delivery,” and “systemic administration,” used interchangeably herein, in some cases can refer to a route of administration of medication, oncolytic virus or other substances into the circulatory system. The systemic administration may comprise intravenous administration, oral administration, intraperitoneal administration, parenteral administration, intranasal administration, sublingual administration, rectal administration, transdermal administration, intra-arterial administration, or any combinations thereof.

### Checkpoint Inhibitors

### Signal Regulatory Protein-Alpha

Immune checkpoint inhibitors block checkpoint proteins from binding their receptor, thereby allowing the immune response to proceed. Use of a checkpoint inhibitor in cancer treatment circumvents a cancer cell's ability to avoid attack by immune cells. Signal Regulatory Protein-alpha (SIRP-alpha) is expressed on phagocytes such as macrophages. The SIRP-alpha protein comprises a cytoplasmic region, a transmembrane region, and three Ig-like domains in an extracellular region. The three Ig-like domains comprise an N-terminal V-like domain and two C1-like Ig domains. The IgV-like SIRP-alpha domain interacts with an IgV-like domain on CD47. This interaction signals a “don't eat me” signal, suppressing phagocytosis of the cell expressing CD47. Inhibition of the CD47-SIRP-alpha interaction allows for phagocytic signaling. Described herein are oncolytic viruses expressing polypeptides with properties that inhibit the CD47-SIRP-alpha checkpoint.

Provided herein, are oncolytic viruses comprising nucleic acids encoding for a variant of SIRP-alpha comprising the IgV domain. Expression of the SIRP-alpha IgV domain provides for a soluble CD47-binding polypeptide. The domain of SIRP-alpha can act as a dominant negative decoy receptor to bind CD47, blocking the CD47-SIRP-alpha signaling pathway (FIG. 1B). Blocking the signaling pathway allows for an increase in macrophage activity.

Provided herein are oncolytic viruses comprising nucleic acids encoding for CD47-SIRP-alpha immune checkpoint inhibitors. In some embodiments, a soluble domain of SIRP-alpha binds CD47 on tumor cells, preventing interaction with SIRP-alpha on macrophages. In some embodiments, the domain of SIRP-alpha comprises a modified domain of SIRP-alpha. In some embodiments, the domain of SIRP-alpha comprises a CD47 binding domain from a SIRP-alpha. In some embodiments, the domain of SIRP-alpha comprises an IgV domain of a SIRP-alpha. In some embodiments, the domain of SIRP-alpha does not comprise a transmembrane domain. In some embodiments, the domain of SIRP-alpha is from a murine SIRP-alpha. In some embodiments, the domain of SIRP-alpha is from a human SIRP-alpha. Exemplary nucleic acid sequences encoding domains of SIRP-alpha are described by SEQ ID NOs: 1-9 as listed in Table 1.

TABLE 1
Nucleic Acid Sequences of SIRP-alpha

| SEQ | | |
|---|---|---|
| ID | | |
| NO: | Variant | Amino Acid Sequence |
| 1 | murine native | ATGTGCCACCAGCAGCTGGTGATCAGCTGGTTCAGCCTGGTGTT |
| | SIRP-alpha | CCTGGCCAGCCCCCTGGTGGCCATCTGGGAGCTGAAGAAGGAC |
| | | GTGTACGTGGTGGAGCTGGACTGGTACCCCGACGCCCCCGGCGA |
| | | GATGGTGGTGCTGACCTGCGACACCCCCGAGGAGGACGGCATC |
| | | ACCTGGACCCTGGACCAGAGCAGCGAGGTGCTGGGCAGCGGCA |
| | | AGACCCTGACCATCCAGGTGAAGGAGTTCGGCGACGCCGGCCA |
| | | GTACACCTGCCACAAGGGCGGCGAGGTGCTGAGCCACAGCCTG |
| | | CTGCTGCTGCACAAGAAGGAGGACGGCATCTGGAGCACCGACA |
| | | TCCTGAAGGACCAGAAGGAGCCCAAGAACAAGACCTTCCTGAG |
| | | GTGCGAGGCCAAGAACTACAGCGGCAGGTTCACCTGCTGGTGG |
| | | CTGACCACCATCAGCACCGACCTGACCTTCAGCGTGAAGAGCAG |
| | | CAGGGGCAGCAGCGACCCCCAGGGCGTGACCTGCGGCGCCGCC |
| | | ACCCTGAGCGCCGAGAGGGTGAGGGGCGACAACAAGGAGTACG |
| | | AGTACAGCGTGGAGTGCCAGGAGGACAGCGCCTGCCCCGCCGC |
| | | CGAGGAGAGCCTGCCCATCGAGGTGATGGTGGACGCCGTGCAC |
| | | AAGCTGAAGTACGAGAACTACACCAGCAGCTTCTTCATCAGGGA |
| | | CATCATCAAGCCCGACCCCCCCAAGAACCTGCAGCTGAAGCCCC |
| | | TGAAGAACAGCAGGCAGGTGGAGGTGAGCTGGGAGTACCCCGA |
| | | CACCTGGAGCACCCCCCACAGCTACTTCAGCCTGACCTTCTGCG |
| | | TGCAGGTGCAGGGCAAGAGCAAGAGGGAGAAGAAGGACAGGG |
| | | TGTTCACCGACAAGACCAGCGCCACCGTGATCTGCAGGAAGAA |
| | | CGCCAGCATCAGCGTGAGGGCCCAGGACAGGTACTACAGCAGC |
| | | AGCTGGAGCGAGTGGGCCAGCGTGCCCTGCAGC |
| 2 | domain of | ATGGAGCCCGCCGGCCCCGCCCCCGGCAGGCTGGGCCCCCTGCT |
| | murine SIRP- | GCTGTGCCTGCTGCTGAGCGCCAGCTGCTTCTGCACCGGCGCCA |
| | alpha | CCGGCAAGGAGCTGAAGGTGACCCAGCCCGAGAAGAGCGTGAG |
| | | CGTGGCCGCCGGCGACAGCACCGTGCTGAACTGCACCCTGACCA |
| | | GCCTGCTGCCCGTGGGCCCCATCAGGTGGTACAGGGGCGTGGGC |
| | | CCCAGCAGGCTGCTGATCTACAGCTTCGCCGGCGAGTACGTGCC |
| | | CAGGATCAGGAACGTGAGCGACACCACCAAGAGGAACAACATG |
| | | GACTTCAGCATCAGGATCAGCAACGTGACCCCCGCCGACGCCGG |
| | | CATCTACTACTGCGTGAAGTTCCAGAAGGGCAGCAGCGAGCCCG |
| | | ACACCGAGATCCAGAGCGGCGGCGGCACCGAGGTG |
| 3 | murine SIRP- | ATGGAGCCCGCCGGCCCCGCCCCCGGCAGGCTGGGCCCCCTGCT |
| | alpha signal | GCTGTGCCTGCTGCTGAGCGCCAGCTGCTTCTGCACCGGCGCCA |
| | sequence | CC |
| 4 | murine SIRP- | GGCAAGGAGCTGAAGGTGACCCAGCCCGAGAAGAGCGTGAGCG |
| | alpha IgV | TGGCCGCCGGCGACAGCACCGTGCTGAACTGCACCCTGACCAGC |
| | domain | CTGCTGCCCGTGGGCCCCATCAGGTGGTACAGGGGCGTGGGCCC |
| | | CAGCAGGCTGCTGATCTACAGCTTCGCCGGCGAGTACGTGCCCA |
| | | GGATCAGGAACGTGAGCGACACCACCAAGAGGAACAACATGGA |
| | | CTTCAGCATCAGGATCAGCAACGTGACCCCCGCCGACGCCGGCA |
| | | TCTACTACTGCGTGAAGTTCCAGAAGGGCAGCAGCGAGCCCGAC |
| | | ACCGAGATCCAGAGC |
| 5 | human native | ATGGAGCCCGCCGGCCCCGCCCCCGGCAGGCTGGGCCCCCTGCT |
| | SIRP-alpha | GTGCCTGCTGCTGGCCGCCAGCTGCGCCTGGAGCGGCGTGGCCG |
| | | GCGAGGAGGAGCTGCAGGTGATCCAGCCCGACAAGAGCGTGCT |
| | | GGTGGCCGCCGGCGAGACCGCCACCCTGAGGTGCACCGCCACC |
| | | AGCCTGATCCCCGTGGGCCCCATCCAGTGGTTCAGGGGCGCCGG |
| | | CCCCGGCAGGGAGCTGATCTACAACCAGAAGGAGGGCCACTTC |
| | | CCCAGGGTGACCACCGTGAGCGACCTGACCAAGAGGAACAACA |
| | | TGGACTTCAGCATCAGGATCGGCAACATCACCCCCGCCGACGCC |
| | | GGCACCTACTACTGCGTGAAGTTCAGGAAGGGCAGCCCCGACG |
| | | ACGTGGAGTTCAAGAGCGGCGCCGGCACCGAGCTGAGCGTGAG |
| | | GGCCAAGCCCAGCGCCCCCGTGGTGAGCGGCCCCGCCGCCAGG |
| | | GCCACCCCCCAGCACACCGTGAGCTTCACCTGCGAGAGCCACGG |
| | | CTTCAGCCCCAGGGACATCACCCTGAAGTGGTTCAAGAACGGCA |
| | | ACGAGCTGAGCGACTTCCAGACCAACGTGGACCCCGTGGGCGA |
| | | GAGCGTGAGCTACAGCATCCACAGCACCGCCAAGGTGGTGCTG |
| | | ACCAGGGAGGACGTGCACAGCCAGGTGATCTGCGAGGTGGCCC |
| | | ACGTGACCCTGCAGGGCGACCCCCTGAGGGGCACCGCCAACCT |
| | | GAGCGAGACCATCAGGGTGCCCCCCACCCTGGAGGTGACCCAG |
| | | CAGCCCGTGAGGGCCGAGAACCAGGTGAACGTGACCTGCCAGG |
| | | TGAGGAAGTTCTACCCCCAGAGGCTGCAGCTGACCTGGCTGGAG |
| | | AACGGCAACGTGAGCAGGACCGAGACCGCCAGCACCGTGACCG |
| | | AGAACAAGGACGGCACCTACAACTGGATGAGCTGGCTGCTGGT |
| | | GAACGTGAGCGCCCACAGGGACGACGTGAAGCTGACCTGCCAG |
| | | GTGGAGCACGACGGCCAGCCCGCCGTGAGCAAGAGCCACGACC |
| | | TGAAGGTGAGCGCCCACCCCAAGGAGCAGGGCAGCAACACCGC |
| | | CGCCGAGAACACCGGCAGCAACGAGAGGAACATCTACATCGTG |
| | | GTGGGCGTGGTGTGCACCCTGCTGGTGGCCCTGCTGATGGCCGC |
| | | CCTGTACCTGGTGAGGATCAGGCAGAAGAAGGCCCAGGGCAGC |
| | | ACCAGCAGCACCAGGCTGCACGAGCCCGAGAAGAACGCCAGGG |
| | | AGATCACCCAGGACACCAACGACATCACCTACGCCGACCTGAA |
| | | CCTGCCCAAGGGCAAGAAGCCCGCCCCCCAGGCCGCCGAGCCC |
| | | AACAACCACACCGAGTACGCCAGCATCCAGACCAGCCCCCAGC |
| | | CCGCCAGCGAGGACACCCTGACCTACGCCGACCTGGACATGGTG |
| | | CACCTGAACAGGACCCCCAAGCAGCCCGCCCCCAAGCCCGAGC |
| | | CCAGCTTCAGCGAGTACGCCAGCGTGCAGGTGCCCAGGAAG |
| 6 | domain of | ATGGAGCCCGCCGGCCCCGCCCCCGGCAGGCTGGGCCCCCTGCT |
| | human SIRP- | GTGCCTGCTGCTGGCCGCCAGCTGCGCCTGGAGCGGCGTGGCCG |
| | alpha | GCGAGGAGGAGCTGCAGGTGATCCAGCCCGACAAGAGCGTGCT |
| | | GGTGGCCGCCGGCGAGACCGCCACCCTGAGGTGCACCGCCACC |
| | | AGCCTGATCCCCGTGGGCCCCATCCAGTGGTTCAGGGGCGCCGG |
| | | CCCCGGCAGGGAGCTGATCTACAACCAGAAGGAGGGCCACTTC |
| | | CCCAGGGTGACCACCGTGAGCGACCTGACCAAGAGGAACAACA |
| | | TGGACTTCAGCATCAGGATCGGCAACATCACCCCCGCCGACGCC |
| | | GGCACCTACTACTGCGTGAAGTTCAGGAAGGGCAGCCCCGACG |
| | | ACGTGGAGTTCAAGAGCGGCGCCGGCACCGAGCTGAGC |
| 7 | human SIRP- | ATGGAGCCCGCCGGCCCCGCCCCCGGCAGGCTGGGCCCCCTGCT |
| | alpha, signal | GTGCCTGCTGCTGGCCGCCAGCTGCGCCTGGAGCGGCGTGGCCG |
| | sequence | GC |
| 8 | human SIRP- | GAGGAGCTGCAGGTGATCCAGCCCGACAAGAGCGTGCTGGTGG |
| | alpha, IgV | CCGCCGGCGAGACCGCCACCCTGAGGTGCACCGCCACCAGCCTG |
| | domain | ATCCCCGTGGGCCCCATCCAGTGGTTCAGGGGCGCCGGCCCCGG |
| | | CAGGGAGCTGATCTACAACCAGAAGGAGGGCCACTTCCCCAGG |
| | | GTGACCACCGTGAGCGACCTGACCAAGAGGAACAACATGGACT |
| | | TCAGCATCAGGATCGGCAACATCACCCCCGCCGACGCCGGCACC |
| | | TACTACTGCGTGAAGTTCAGGAAGGGCAGCCCCGACGACGTGG |
| | | AGTTCAAGAGCGGC |
| 9 | domain of | GCCACCAGGACCGAGGTGAAGGTGATCCAGCCCGAGAAGAGCG |
| | mouse NOD | TGAGCGTGGCCGCCGGCGACAGCACCGTGCTGAACTGCACCCTG |
| | SIRP-alpha | ACCAGCCTGCTGCCCGTGGGCCCCATCAGGTGGTACAGGGGCGT |
| | | GGGCCAGAGCAGGCAGCTGATCTACAGCTTCACCACCGAGCACT |
| | | TCCCCAGGGTGACCAACGTGAGCGACGCCACCAAGAGGAGCAA |
| | | CCTGGACTTCAGCATCAGGATCAGCAACGTGACCCCCGAGGACG |
| | | CCGGCACCTACTACTGCGTGAAGTTCCAGAGGGGCAGCCCCGAC |
| | | ACCGAGATCCAGAGCGGCGGCGGCACCGAGGTGTACGTGCTGG |
| | | CC |

Provided here are oncolytic viruses comprising nucleic acids encoding for a domain of a murine SIRP-alpha. In some embodiments, the nucleic acid has a sequence as in SEQ ID NO: 2. In some embodiments, the nucleic acid encoding a domain of murine SIRP-alpha comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 2.

Provided herein are oncolytic viruses comprising nucleic acids encoding for a domain of a human SIRP-alpha. In some embodiments, the nucleic acid has a sequence as in SEQ ID NO: 6. In some embodiments, the nucleic acid encoding a domain of human SIRP-alpha comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 6.

Provided herein are compositions comprising nucleic acids encoding for a domain of non-obese diabetic (NOD) mouse SIRP-alpha. In some embodiments, the nucleic acid has a sequence as in SEQ ID NO: 9. In some embodiments, the nucleic acid encoding a domain of NOD murine SIRP-alpha comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 9.

Native SIRP-alpha is a membrane-bound 513 (murine) or 504 (human) amino acid protein. Domains starting at the amino terminus include an IgV-like CD47-binding domain and two C1-like Ig domains in the extracellular region, a transmembrane domain, and a cytoplasmic domain. The IgV-like CD47-binding domain in murine or human SIRP-alpha comprises 143 amino acids as described by SEQ ID NO: 13 or SEQ ID NO: 17. Exemplary amino acid sequences expressed by nucleic acids as described by SEQ ID NOs: 10-18 are listed in Table 2.

TABLE 2
Amino Acid Sequences of SIRP-alpha

| SEQ | | UniProtKB | |
|---|---|---|---|
| ID | | accession | |
| NO: | Variant | ID | Amino Acid Sequence |
| 10 | murine native | P97797 | MEPAGPAPGRLGPLLLCLLLSASCFCTGATGKELKV |
| | SIRP-alpha | | TQPEKSVSVAAGDSTVLNCTLTSLLPVGPIRWYRGV |
| | | | GPSRLLIYSFAGEYVPRIRNVSDTTKRNNMDFSIRIS |
| | | | NVTPADAGIYYCVKFQKGSSEPDTEIQSGGGTEVYV |
| | | | LAKPSPPEVSGPADRGIPDQKVNFTCKSHGFSPRNIT |
| | | | LKWFKDGQELHPLETTVNPSGKNVSYNISSTVRVVL |
| | | | NSMDVNSKVICEVAHITLDRSPLRGIANLSNFIRVS |
| | | | PTVKVTQQSPTSMNQVNLTCRAERFYPEDLQLIWL |
| | | | ENGNVSRNDTPKNLTKNTDGTYNYTSLFLVNSSAH |
| | | | REDVVFTCQVKHDQQPAITRNHTVLGFAHSSDQGS |
| | | | MQTFPDNNATHNWNVFIGVGVACALLVVLLMAALY |
| | | | LLRIKQKKAKGSTSSTRLHEPEKNAREITQVQSLIQ |
| | | | DTNDINDITYADLNLPKEKKPAPRAPEPNNHTEYAS |
| | | | IETGKVPRPEDTLTYADLDMVHLSRAQPAPKPEPSF |
| | | | SEYASVQVQRK |
| 11 | domain of | | MEPAGPAPGRLGPLLLCLLLSASCFCTGATGKELKV |
| | murine SIRP- | | TQPEKSVSVAAGDSTVLNCTLTSLLPVGPIRWYRGV |
| | alpha | | GPSRLLIYSFAGEYVPRIRNVSDTTKRNNMDFSIRIS |
| | | | NVTPADAGIYYCVKFQKGSSEPDTEIQSGGGTEV |
| 12 | murine SIRP- | | MEPAGPAPGRLGPLLLCLLLSASCFCTGATG |
| | alpha signal | | |
| | sequence | | |
| 13 | murine SIRP- | | KELKVTQPEKSVSVAAGDSTVLNCTLTSLLPVGPIR |
| | alpha Ig like | | WYRGVGPSRLLIYSFAGEYVPRIRNVSDTTKRNNM |
| | V-type | | DFSIRISNVTPADAGIYYCVKFQKGSSEPDTEIQS |
| | domain | | |
| 14 | human native | P78324.1 | MEPAGPAPGRLGPLLCLLLAASCAWSGVAGEEELQ |
| | SIRP-alpha | | VIQPDKSVLVAAGETATLRCTATSLIPVGPIQWFRG |
| | | | AGPGRELIYNQKEGHFPRVTTVSDLTKRNNMDFSIR |
| | | | IGNITPADAGTYYCVKFRKGSPDDVEFKSGAGTELS |
| | | | VRAKPSAPVVSGPAARATPQHTVSFTCESHGFSPRD |
| | | | ITLKWFKNGNELSDFQTNVDPVGESVSYSIHSTAKV |
| | | | VLTREDVHSQVICEVAHVTLQGDPLRGTANLSETIR |
| | | | VPPTLEVTQQPVRAENQVNVTCQVRKFYPQRLQLT |
| | | | WLENGNVSRTETASTVTENKDGTYNWMSWLLVN |
| | | | VSAHRDDVKLTCQVEHDGQPAVSKSHDLKVSAHP |
| | | | KEQGSNTAAENTGSNERNIYIVVGVVCTLLVALLM |
| | | | AALYLVRIRQKKAQGSTSSTRLHEPEKNAREITQDT |
| | | | NDITYADLNLPKGKKPAPQAAEPNNHTEYASIQTSP |
| | | | QPASEDTLTYADLDMVHLNRTPKQPAPKPEPSFSEY |
| | | | ASVQVPRK |
| 15 | domain of | | MEPAGPAPGRLGPLLCLLLAASCAWSGVAGEEELQ |
| | human SIRP- | | VIQPDKSVLVAAGETATLRCTATSLIPVGPIQWFRG |
| | alpha | | AGPGRELIYNQKEGHFPRVTTVSDLTKRNNMDFSIR |
| | | | IGNITPADAGTYYCVKFRKGSPDDVEFKSGAGTELS |
| 16 | human SIRP- | | MEPAGPAPGRLGPLLCLLLAASCAWSGVAG |
| | alpha, signal | | |
| | sequence | | |
| 17 | human SIRP- | | EELQVIQPDKSVLVAAGETATLRCTATSLIPVGPIQ |
| | alpha, Ig like | | WFRGAGPGRELIYNQKEGHFPRVTTVSDLTKRNNM |
| | V-type | | DFSIRIGNITPADAGTYYCVKFRKGSPDDVEFKSG |
| | domain | | |
| 18 | domain of | | MEPAGPAPGRLGPLLCLLLAASCAWSGVAGETEVK |
| | NOD mouse | | VIQPEKSVSVAAGDSTVLNCTLTSLLPVGPIRWYRG |
| | SIRP-alpha | | VGQSRQLIYSFTTEHFPRVTNVSDATKRSNLDFSIRI |
| | | | SNVTPEDAGTYYCVKFQRGSPDTEIQSGGGTEVYV |
| | | | LAK |

Provided here are oncolytic viruses comprising nucleic acids encoding for a domain of a murine SIRP-alpha. In some embodiments, the nucleic acid sequence encodes for a peptide described by SEQ ID NO: 11. In some embodiments, the encoded domain of murine SIRP-alpha comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 11.

Provided here are oncolytic viruses comprising nucleic acids encoding for a domain of a human SIRP-alpha. In some embodiments, the nucleic acid sequence encodes for a peptide described by SEQ ID NO: 15. In some embodiments, the encoded domain of human SIRP-alpha comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 15.

Provided here are oncolytic viruses comprising nucleic acids encoding for a domain of a non-obese diabetic (NOD) mouse SIRP-alpha. In some embodiments, the domain of NOD mouse SIRP-alpha encodes for a peptide described by SEQ ID NO: 18. In some embodiments, the encoded domain of NOD mouse SIRP-alpha comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 18.

### Other Immune Checkpoint Inhibitors

Antibody dependent cellular cytotoxicity (ADCC) and antibody dependent cellular phagocytosis (ADCP) are induced by binding of an antibody Fc region with Fc (gamma) R on effector cells. The Fc region can be from any type of antibody, for example an Immunoglobulin G (IgG) antibody. An IgG antibody can be subclass 1, 2, 3, or 4. In some cases, an antibody, monoclonal antibody, or humanized monoclonal antibody has antigen specificity to CD47 or SIRP-alpha and acts as an immune checkpoint inhibitor. In some cases, an Fc region is fused with an immune checkpoint inhibitor as described herein. The combination of immune checkpoint inhibitor and Fc region can provide for induction of phagocytosis and cytolysis in addition to the “don't eat me” signal inhibition.

Provided here are oncolytic viruses comprising nucleic acids encoding for a SIRP-alpha-Fc fusion protein. In some embodiments the SIRP-alpha-Fc fusion comprises a human SIRP-alpha IgV-IgG1 Fc fusion. In some embodiments, the human SIRP-alpha IgV-IgG1 Fc fusion comprises the amino acid sequence as in SEQ ID NO 19. In some embodiments, the human SIRP-alpha IgV-IgG1 Fc fusion comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 19. In some embodiments the SIRP-alpha-Fc fusion comprises a human SIRP-alpha IgV-IgG4 Fc fusion. In some embodiments, the human SIRP-alpha IgV-IgG4 Fc fusion comprises the amino acid sequence as in SEQ ID NO 20. In some embodiments, the human SIRP-alpha IgV-IgG4 Fc fusion comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 20.

Provided herein are oncolytic viruses comprising nucleic acids encoding for an anti-CD47 antibody. In some embodiments, the antibody is a monoclonal antibody. In some embodiments, the monoclonal antibody is a humanized monoclonal antibody. In some embodiments, the humanized monoclonal antibody comprises the heavy chain sequence of SEQ ID NO: 21. In some embodiments, the humanized monoclonal antibody comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 21. In some embodiments, the humanized monoclonal antibody comprises the light chain sequence of SEQ ID NO: 22. In some embodiments, the humanized monoclonal antibody comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 22. In some embodiments, the anti-CD47 antibody is magrolimab. Sequences of described fusion proteins and antibodies are listed in Table 3.

TABLE 3
Amino acid sequences of antibody subunit proteins

| SEQ | | | |
|---|---|---|---|
| ID | | | |
| NO: | Description | ID number | Amino Acid Sequence |
| 19 | Human | UniProtKB | EEELQVIQPDKSVSVAAGESAILHCTVTSLIPVGPIQ |
| | SIRP-alpha | P01857 | WFRGAGPARELIYNQKEGHFPRVTTVSESTKRENMDF |
| | IgV-IgG1 Fc | | SISISNITPADAGTYYCVKFRKGSPDTEFKSGASTKG |
| | (TT-621) | | PSVFPLAPSSKSTSGGTAALGCLVKDYFPEPVTVSWN |
| | IgG1 Fc in | | SGALTSGVHTFPAVLQSSGLYSLSSVVTVPSSSLGTQ |
| | bold | | TYICNVNHKPSNTKVDKKVEPKSCDKTHTCPPCPAPE |
| | | | LLGGPSVFLFPPKPKDTLMISRTPEVTCVVVDVSHED |
| | | | PEVKFNWYVDGVEVHNAKTKPREEQYNSTYRVVSVLT |
| | | | VLHQDWLNGKEYKCKVSNKALPAPIEKTISKAKGQPR |
| | | | EPQVYTLPPSRDELTKNQVSLTCLVKGFYPSDIAVEW |
| | | | ESNGQPENNYKTTPPVLDSDGSFFLYSKLTVDKSRWQ |
| | | | QGNVFSCSVMHEALHNHYTQKSLSLSPGK |
| 20 | Human | CAS ID | EEELQVIQPDKSVSVAAGESAILHCTVTSLIPVGPIQWF |
| | SIRP-alpha | 2631667-06-4 | RGAGPARELIYNQKEGHFPRVTTVSESTKRENMDFSISI |
| | IgV-IgG4 Fc | | SNITPADAGTYYCVKFRKGSPDTEFKSGAGTELSVRA |
| | (TT-622) | | KPSESKYGPPCPPCPAPEFLGGPSVFLFPPKPKDTL |
| | IgG4 Fc in | | MISRTPEVTCVVVDVSQEDPEVQFNWYVDGVEVHN |
| | bold | | AKTKPREEQFNSTYRVVSVLTVLHQDWLNGKEYKC |
| | | | KVSNKGLPSSIEKTISKAKGQPREPQVYTLPPSQE |
| | | | EMTKNQVSLTCLVKGFYPSDIAVEWESNGQPENNY |
| | | | KTTPPVLDSDGSFFLYSRLTVDKSRWQEGNVFSCS |
| | | | VMHEALHNHYTQKSLSLSLGK |
| 21 | Magrolimab | CAS: | QVQLVQSGAEVKKPGASVKVSCKASGYTFTNYNMH |
| | Heavy chain: | 2169232-81-7 | WVRQAPGQRLEWMGTIYPGNDDTSYNQKFKDRVTIT |
| | HU5F9-G4 | | ADTSASTAYMELSSLRSEDTAVYYCARGGYRAMDYW |
| | | | GQGTLVTVSSASTKGPSVFPLAPCSRSTSESTAALG |
| | | | CLVKDYFPEPVTVSWNSGALTSGVHTFPAVLQSSGL |
| | | | YSLSSVVTVPSSSLGTKTYTCNVDHKPSNTKVDKRV |
| | | | ESKYGPPCPPCPAPEFLGGPSVFLFPPKPKDTLMIS |
| | | | RTPEVTCVVVDVSQEDPEVQFNWYVDGVEVHNAKTK |
| | | | PREEQFNSTYRVVSVLTVLHQDWLNGKEYKCKVSNK |
| | | | GLPSSIEKTISKAKGQPREPQVYTLPPSQEEMTKNQ |
| | | | VSLTCLVKGFYPSDIAVEWESNGQPENNYKTTPPVL |
| | | | DSDGSFFLYSRLTVDKSRWQEGNVFSCSVMHEALHN |
| | | | HYTQKSLSLSLGK |
| 22 | Magrolimab | CAS: | DIVMTQSPLSLPVTPGEPASISCRSSQSIVYSNGNTYLG |
| | Light chain | 2169232-81-7 | WYLQKPGQSPQLLIYKVSNRFSGVPDRFSGSGSGTDFT |
| | | | LKISRVEAEDVGVYYCFQGSHVPYTFGQGTKLEIKRTV |
| | | | AAPSVFIFPPSDEQLKSGTASVVCLLNNFYPREAKVQW |
| | | | KVDNALQSGNSQESVTEQDSKDSTYSLSSTLTLSKAD |
| | | | YEKHKVYACEVTHQGLSSPVTKSFNRGEC |

### Promoters

Provided herein are oncolytic viruses comprising nucleic acids, wherein the nucleic acid encodes for at least one promoter region. A promoter region, or promoter, or promoter element, or regulatory region, refers to a nucleic acid sequence to which proteins bind to initiate transcription. Promoters are typically located 5′, or upstream, to a DNA coding region which they control. In some embodiments, a nucleic acid described herein comprises one promoter. In some embodiments, the one promoter drives transcription of all polypeptides encoded on the nucleic acid. In some embodiments, a nucleic acid described herein comprises a separate promoter for each polypeptide encoded on the nucleic acid. In some embodiments, the nucleic acid comprises two promoters, each driving transcription of one of two polypeptides encoded on the nucleic acid.

Timing of expression can be modulated by the structure of the promotor regulating the gene expression. The number and affinity of transcription factor binding sites determines the relative timing of expression between different promoter regions. A promoter with more transcription factor binding sites and/or higher binding affinity can drive expression earlier than a promoter with fewer or lower affinity binding sites.

Application of the relative temporal expression of proteins, can be leveraged to express particular factors from modified viruses as described herein either earlier or later in the infection process. In some embodiments, a receptor is expressed using an early promoter. Expression early in infection allows for expression and processing by the cell, before cellular processes are disrupted. In some embodiments, one or more cytokines are expressed using a late promoter.

Provided herein, in some embodiments, is one or promoters comprising P7.5, P10, P28, P135, TK promoter, A52R promoter, 454 promoter, PB8, LEO, PF11, F7L, H5R, mH5, H1L, A1L, J3R, E4L, I1L, I3L, I4L, 15L, I7L, T7, I2L, FP4b, ATI, P11, PFL1, L4R, T7 promoter, 28 kDa promoter, a short synthetic promoter (SSP), or any functional variant or combination thereof. In some embodiments, the promoter is an early promoter. In some embodiments, the promoter comprises an early promoter or a late promoter. In some embodiments, the early promoter comprises A52R, PB8, mH5, I4L, LEO, PF11, I3L, P7.5, TK promoter, F7L, H5R, a short synthetic promoter (SSP) or any variation or combination thereof. In some embodiments, the late promoter comprises SSP, P7.5, P28, P135, TK promoter, F7L, H5R, H1L, A1L, J3R, E4L, I1L, I5L, I7L, T7, I2L, FP4b, ATI, P11, PFL1, L4R, 28 kDa promoter or any functional variant or combination thereof. Sequences of selected promoters are listed in Table 4.

TABLE 4
Promoter nucleic acid sequences

| SEQ ID | | |
|---|---|---|
| NO: | Description | Nucleic Acid Sequence |
| 23 | Vaccinia P7.5 | TCACTAATTCCAAACCCACCCGCTTTTTATAGTAAGTTTTTCAC |
| | | CCATAAATAATAAATACAATAATTAATTTCTCGTAAAAGTAGA |
| | | AAATATATTCTAATTTATTGCACGGTAAGGAAGTAGATCATAA |
| 24 | P10 promoter | AAAAATTGAAAAACTAGCGTCTTTTTTTGCTCGA |
| 25 | P28 promoter | AAGCTCACAAAAAAAACTTCTCTAAATAGATTCGAGC |
| 26 | 454 promoter | GGATCATAAAAATATAGTAGAATTTCATTTTGTTTTTTTCTATG |
| | | CTATAAATAGCTTCTCGAGC |
| 27 | VACWR135 | CCTGCAGGGCGTCCTAAAATTAAAACAAATGATATAA |
| | (P135) | |
| 28 | Strong | GTGATGCGGTTTTGGCAGTACATCAATGGGCGTGGATAGCGGT |
| | vaccinia virus | TTGACTCACGGGGATTTCCAAGTCTCCACCCCATTGACGTCAA |
| | early promoter | TGGGAGTTTGTTTTGGCACCAAAATCAACGGGACTTTCCAAAA |
| | | TGTCGTAACAACTCCGCCCCATTGACGCAAATGGGCGGTAGG |
| | | CGTGTACGGTGGGAGGTCTATATAAGCAGAGCT |
| 29 | Human PGK | GGGGTTGGGGTTGCGCCTTTTCCAAGGCAGCCCTGGGTTTGCG |
| | promoter | CAGGGACGCGGCTGCTCTGGGCGTGGTTCCGGGAAACGCAGC |
| | | GGCGCCGACCCTGGGTCTCGCACATTCTTCACGTCCGTTCGCA |
| | | GCGTCACCCGGATCTTCGCCGCTACCCTTGTGGGCCCCCCGGC |
| | | GACGCTTCCTGCTCCGCCCCTAAGTCGGGAAGGTTCCTTGCGG |
| | | TTCGCGGCGTGCCGGACGTGACAAACGGAAGCCGCACGTCTC |
| | | ACTAGTACCCTCGCAGACGGACAGCGCCAGGGAGCAATGGCA |
| | | GCGCGCCGACCGCGATGGGCTGTGGCCAATAGCGGCTGCTCA |
| | | GCAGGGCGCGCCGAGAGCAGCGGCCGGGAAGGGGCGGTGCG |
| | | GGAGGCGGGGTGTGGGGCGGTAGTGTGGGCCCTGTTCCTGCC |
| | | CGCGCGGTGTTCCGCATTCTGCAAGCCTCCGGAGCGCACGTCG |
| | | GCAGTCGGCTCCCTCGTTGACCGAATCACCGACCTCTCTCCCC |
| | | AG |

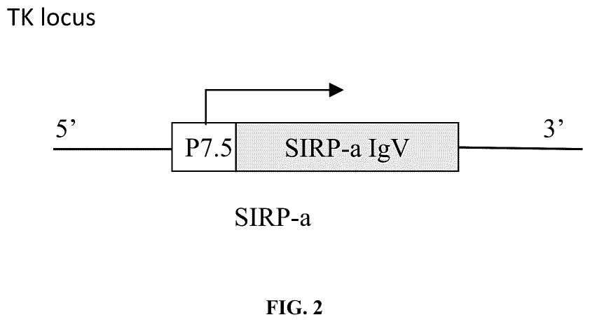

Oncolytic viruses provided herein may comprise a P7.5 promoter. In some embodiments, a P7.5 promoter drives expression of a domain of SIRP-alpha. A schematic representation of promoter and transgene inserted at a TK locus is shown in FIG. 2. In some embodiments, the P7.5 promoter comprises a nucleic acid sequence of SEQ ID NO: 23.

Oncolytic viruses provided herein may comprise a P10 promoter. In some embodiments, the P10 promoter drives expression of a region encoding for a domain of SIRP-alpha. In some embodiments, the P10 promoter comprises a sequence of SEQ ID NO: 24.

Provided herein are oncolytic viruses comprising an exogenous nucleic acid. In some embodiments the exogenous nucleic acid comprises RNA. In some embodiments, the exogenous nucleic acid comprises DNA. In some embodiments, the DNA encodes for a domain of SIRP-alpha. In some embodiments, the DNA comprises, in 5′ to 3′ order, a P7.5 promoter (SEQ ID NO: 23) and a domain of murine SIRP-alpha (SEQ ID NO: 2). In some embodiments, the DNA comprises, in 5′ to 3′ order, a P10 promoter (SEQ ID NO: 24), and murine SIRP-alpha (SEQ ID NO: 2). In some embodiments, the DNA comprises, in 5′ to 3′ order, a P7.5 promoter (SEQ ID NO: 23), and a domain of human SIRP-alpha (SEQ ID NO: 6). In some embodiments, the DNA comprises, in 5′ to 3′ order, a P10 promoter (SEQ ID NO: 24) and a domain of human SIRP-alpha (SEQ ID NO: 6). In some embodiments, exogenous nucleic acids described herein are incorporated into a viral genome.

### Vectors

Provided herein are compositions comprising vectors. Generally, vectors are vehicles designed to carry a nucleic acid into a cell. In some embodiments, the vector comprises a plasmid, a phage, a virus, a cosmid, or an artificial chromosome. For the purposes of this application, a vector can also comprise transfection agents and methods, such as liposomes, nanoparticles, electroporation, microinjection, gene gun, impalefection, hydrostatic pressure, continuous infusion, sonication, or any combination thereof. Provided herein are compositions comprising a vector and one or more nucleic acids as described herein. In some embodiments, the vector comprises a virus. In some embodiments, the virus is a retrovirus, lentivirus, adenovirus, adeno-associated virus, or a herpes simplex virus. In some embodiments, the virus is an oncolytic virus. In some embodiments, the vector is a modified virus. In some embodiments, the virus comprises a mutation or deletion of one or more genes. In some embodiments, the virus comprises one or more exogenous nucleic acids as described herein.

### Oncolytic Viruses

Provided herein are compositions comprising an oncolytic virus. In some embodiments, the oncolytic virus comprises a nucleic acid described herein. Oncolytic viruses, as used herein, kill cancer or tumor cells through mechanisms such as the direct lysis of said cells, by stimulating immune response towards said cells, apoptosis, expression of toxic proteins, autophagy and shutdown of protein synthesis, induction of anti-tumoral immunity, or any combinations thereof. In some embodiments, an oncolytic virus as described herein replicates within a cell. In some embodiments, an oncolytic virus as described herein replicates within a tumor cell, an immune cell, a somatic cell, a hemopoietic cell, or another type of cell. Exemplary oncolytic viruses for inclusion in a composition described herein include, without limitation, a poxvirus, a vaccinia virus, an adeno associated virus, an adenovirus, a reovirus, a lentivirus, a herpes simplex virus, a vesicular stomatitis virus, a mengovirus, a myxoma virus, Newcastle disease virus, a senecavirus, a retrovirus, measles virus, maraba virus, coxsackievirus, or polio virus. These oncolytic viruses have a proclivity to specifically target cancer cells, and upon virus replication cause significant cell death and tumor regression. In some embodiments, the oncolytic virus is a vaccinia virus. Exemplary vaccinia viruses include, without limitation, the following strains for modification by inclusion of an exogenous nucleic acid described herein: Western Reserve Vaccinia virus (ATCC VR-1354), Vaccinia virus Ankara (ATCC VR-1508), Vaccinia virus Ankara (ATCC VR-1566), Vaccinia virus strain Wyeth (ATCC VR-1536), or Vaccinia virus Wyeth (ATCC VR-325). Furthermore, in some embodiments, the recombinant vaccinia viruses are modified versions of a wild type or attenuated vaccinia virus strain. Nonlimiting examples of vaccinia virus strains include a Western Reserve strain of vaccinia virus, a Copenhagen strain, a IHD strain, a Wyeth (NYCBOH) strain, a Tian Tan strain, a Lister strain, a USSR strain, an Ankara strain, an NYVAC strain, an Ankara (MVA) strain, a Paris strain, a Bern strain, a Temple of Heaven strain, a Dairen strain, an EM-63 strain, an Evans strain, a King strain, a Patwadangar strain, or a Tash Kent strain.

Oncolytic viruses are optionally recombinant or selected to have low toxicity and to accumulate in the target tissue. In some embodiments, the modifications in the viral backbone/viral genome are modifications that render the virus non-replicating or comprise a poor replicative capacity. The base oncolytic virus strain modified as set forth herein optionally comprises one or more mutations or one or more deletions relative to its parent strain. In some embodiments, a modification includes mutation or complete or partial deletion in one or more of the following viral genes: A1, A2, VH1, A33, 17, A52R, TK, B15R, K7R, B14R, NIL, K1L, M2L, A49R, A46R, B8R, C12L, B18R, A52R, F3L, C4, or C16. In some embodiments, the viral backbone mutation is selected from the group consisting of: a complete or partial deletion of the A1 gene; a complete or partial deletion of the A2 gene; a complete or partial deletion of the VH1 gene; a complete or partial deletion of the A33 gene; a complete or partial deletion of 17 gene; a complete or partial deletion of the A52R gene; a complete or partial deletion of the TK gene; a complete or partial deletion of the B15R gene; a complete or partial deletion of the K7R gene; a complete or partial deletion of the B14R gene; a complete or partial deletion of the NIL gene; a complete or partial deletion of the K1L gene; a complete or partial deletion of the M2L gene; a complete or partial deletion of the A49R gene; a complete or partial deletion of the A46R gene; a complete or partial deletion of the B8R gene; a complete or partial deletion of the C12L gene; a complete or partial deletion of the B18R gene; a complete or partial deletion of the A52R gene; a complete or partial deletion of the F3L gene; a complete or partial deletion of the C4 gene; a complete or partial deletion of the C16 gene. As used herein, the reference to a viral gene is made by reference to the protein encoded by the gene (e.g., A33 gene means a gene that encodes for the A33 protein). In some embodiments, the viral backbone mutation, including any combinations of substitution, insertion, and deletion, result in a sequence with less than 100%, 99%, 98%, 97%, 96%, 95%, 94%, 93%, 92%, 91%, 90% or less sequence homology to the wild-type sequence of the viral gene or a viral protein encoded by the gene. In some embodiments, the viral backbone comprises 1, 2, 3, 4, 5, or more mutations in the amino acid sequence of the viral protein (e.g., a viral antigen). The disclosure provides in some embodiments, recombinant oncolytic viruses containing one more mutation(s) in the genome of the virus (virus back bone) such that the mutation increases the T-cell arm of the immune response. A mutation may be addition, deletion, or substitution of one or more nucleic acid(s) in the viral genome (wild type or attenuated native strains of oncolytic virus). In non-limiting examples, the mutation is complete or partial deletion of genes that are known to inhibit cytokines involved in the Th1 immune response. In some embodiments, the mutation is a deletion of nucleic acid encoding for B8R (interferon gamma (IFN-g) binding proteins). In some embodiments, the mutation is a deletion of nucleic acid encoding for C12L (interleukin-18 (IL-18) binding proteins). In some embodiments, the mutation is a complete or partial deletion of genes in innate immune signaling. In some embodiments, the mutation is a complete or partial deletion of nucleic acid encoding for B18R (type I interferon (IFN)-binding proteins). In some embodiments, the mutation is a complete or partial deletion of nucleic acid encoding for A52R (nuclear factor κB (NF-κB) inhibitor proteins). In some embodiments, the mutation is a complete or partial deletion of nucleic acid encoding for E3L (protein kinase (PKR) inhibitors), In some embodiments, the mutation is a complete or partial deletion of nucleic acid encoding for C4 or C16 (STING pathway inhibitors.

An oncolytic virus, as described herein, contains one or more additional insertions or partial insertions of exogenous nucleic acids that encode for one or more polypeptides. In some embodiments, the one or more polypeptides comprise a soluble CD47-SIRP-alpha immune checkpoint inhibitor as described herein. An oncolytic virus of the current disclosure further contains one or more additional deletions or partial deletions of one or more genes from TK, A52R, B15R, K7R, A46R, N1L, E3L, K1L, M2L, C16, N2R, B8R, B18R, VH1 and a functional domain or fragment or variant thereof, or any combinations thereof. In some embodiments, the oncolytic virus provided herein contains a complete or partial deletion of the TK viral genes, and insertion of an exogenous nucleic acid encoding for one or more polypeptides. In some embodiments, the oncolytic virus further comprises a full or partial deletion of the B8R gene.

In some embodiments, the oncolytic virus comprises one or more modifications that results in a greater therapeutic effect against tumor cells, as compared to an otherwise identical virus that does not comprise the modifications. In some non-limiting examples, the greater therapeutic effect includes each or any combinations of: enhanced immune evasion of the virus, enhanced tumor-targeted systemic delivery of the virus, enhanced intra-tumoral and intertumoral spreading of the virus, and enhanced tumor-specific replication of the virus, or release of immune modulators and anti-tumor agents into the extracellular matrix. The oncolytic virus of this disclosure, in some instances, is utilized as a platform vector for systemic delivery.

In some embodiments, provided herein is an oncolytic virus comprising a modification that enhances an immune response to a tumor. Oncolytic viruses are optionally (a) administered systemically, (b) inoculated topically over the tumor, or (c) injected directly into the tumor (“intratumoral delivery”). In general, such a virus comprises a modification to its constituent, such as, but not limited to, a modification in the native genome (“backbone”) of the virus like a mutation or a deletion of a viral gene, introduction of an exogenous nucleic acid, a chemical modification of a viral nucleic acid or a viral protein, and introduction of an exogenous protein or modified viral protein to the viral capsid.

In some embodiments, the oncolytic virus comprises a full-length viral backbone gene or viral backbone protein described above, or truncated versions thereof, or functional domains thereof, or fragments thereof, or variants thereof. In various examples, the oncolytic virus comprises a mutation or deletion of one or more of viral backbone genes or viral backbone proteins, as described above. Mutations of the viral backbone genes and viral backbone proteins comprise insertion, deletion, substitution, or modifications of nucleotides in nucleic acid sequences and amino acids in protein sequences. Deletion comprises, in some examples, a complete or partial deletion of the viral backbone gene or protein.

In some embodiments, the modification of the oncolytic virus results in at least about 1.1, 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2, 6.5, 6.8, 7, 7.2, 7.5, 7.8, 8, 8.2, 8.5, 8.8, 9, 9.2, 9.5, 9.8, 10, 12, 14, 15, 16, 18, 20, 25, 30, 35, 40, 45, 50, 55, 60, 65, 70, 75, 80, 85, 90, 95, 100, 150, 200, 250, 500, 800, 1000, 2500, 5000, 10⁴, 2.5×10⁴, 5×10⁴, 7.5×10⁴, 2.5×10⁵, 5×10⁵, 7.5×10⁵, 10⁶, 2.5×10⁶, 5×10⁶, 7.5×10⁶, 10⁷, 2.5×10⁷, 5×10⁷, 7.5×10⁷, 10⁸, 2.5×10⁸, 5×10⁸, 7.5×10⁸, 10⁹, 2.5×10⁹, 5×10⁹, 7.5×10⁹, 10¹⁰ or even more folds increase in the efficacy of tumor-targeted systemic delivery of the virus, as compared to an otherwise identical oncolytic virus that does not comprise the modification. In certain embodiments, the efficacy of tumor-targeted systemic delivery of the virus is measured by quantifying the viruses infecting the tumor cells, and optionally, in contrast with the viruses infecting non-tumor cells in the body. For instance, in some cases, the quantification of the virus is performed by staining the viral particles in tissue sections, or blood smear in the cases of leukemia, lymphoma, or myeloma. In some embodiments, such quantification is performed by reporter molecule(s) that is/are engineered to be expressed by the viruses, e.g., luciferase, and fluorescent proteins. In some cases, such quantification is performed by quantifying the viral genome in the tumor. Without being limited, it is also possible to measure the tumor-targeted systemic delivery of the virus by quantifying certain downstream effect(s) of viral infection in tumor cells, like cytokines in response to viral infection or lymphocyte accumulation.

In some embodiments, provided herein is an oncolytic virus comprising an exogenous nucleic acid that encodes for a soluble immune checkpoint inhibitor. Expression of the soluble immune checkpoint inhibitor by the oncolytic virus results in boosted immune responses against the infected tumor. Following infection, the oncolytic virus replicates in the tumor cells resulting in expression of the soluble immune checkpoint inhibitor in the tumor environment. These soluble inhibitors function as decoy receptors, binding to the checkpoint ligand, blocking the immune response inhibition in the tumor. Consequently, the immunosuppressive microenvironment in the tumor is altered, leading to enhanced immunotherapeutic activity of the oncolytic virus, as compared to an otherwise identical virus that does not comprise the nucleic acid encoding for the immune checkpoint inhibitor. In some embodiments, the increase in immunotherapeutic activity is at least about 1.1, 1.1, 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2, 6.5, 6.8, 7, 7.2, 7.5, 7.8, 8, 8.2, 8.5, 8.8, 9, 9.2, 9.5, 9.8, 10, 12, 14, 15, 16, 18, 20, 25, 30, 35, 40, 45, 50, 55, 60, 65, 70, 75, 80, 85, 90, 95, 100, 150, 200, 250, 500, 800, 1000, 2500, 5000, 10⁴, 2.5×10⁴, 5×10⁴, 7.5×10⁴, 2.5×10⁵, 5×10⁵, 10⁶ or even higher folds. Without being limited, the increased immunotherapeutic activity is reflected by increased B cell accumulation in the tumor, increased T cell response to tumor-related immunogens, increased macrophage activity in the tumor, or any combination thereof. B cell accumulation is measured, for example, by quantifying the B cells in the tumor, and T cell immuno-activity is measured by, for example, interferon-y (interferon-gamma) secretion in ELISPOT assays.

### Conditions for Treatment

Provided herein are methods for treatment of cancer including administration of a composition described herein. In some embodiments, the method of treatment is for a hyperproliferative disease. In some embodiments, the hyperproliferative disease is a cancer. In some embodiments, the hyperproliferative disease comprises a tumor. Treatments comprising delivery of an oncolytic virus as described herein is contemplated. In some embodiments, the cancer is melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, a myeloproliferative neoplasm, or sarcoma.

In some embodiments, compositions described herein are administered to cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer is optionally of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchio-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; or hairy cell leukemia. In some cases, solid cancers that are metastatic are treated using the oncolytic viruses of this disclosure, such as an oncolytic virus that is advantageous for systemic delivery. In some cases, solid cancers that are inaccessible or difficult to access, such as for purpose of intratumoral delivery of therapeutic agents, are treated using the oncolytic viruses of this disclosure that is advantageous for systemic delivery.

This disclosure also contemplates methods for inhibiting or preventing local invasiveness or metastasis, or both, of any type of primary cancer. In exemplary embodiments, the primary cancer is melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder. In certain embodiments, the primary cancer is lung cancer. For example, the lung cancer is non-small cell lung carcinoma. Moreover, this disclosure optionally is used to prevent cancer or to treat pre-cancers or premalignant cells, including metaplasias, dysplasias, and hyperplasias. It can also be used to inhibit undesirable but benign cells, such as squamous metaplasia, dysplasia, benign prostate hyperplasia cells, hyperplastic lesions, and the like. In some embodiments, the progression to cancer or to a more severe form of cancer is halted, disrupted, or delayed by methods of this disclosure involving an oncolytic virus as described herein.

Provided herein are methods for treating a subject by administration of one or more modified oncolytic viruses, as disclosed herein. An “individual” or “subject,” as used interchangeably herein, refers to a human or a non-human subject. Non-limiting examples of non-human subjects include non-human primates, dogs, cats, mice, rats, guinea pigs, rabbits, pigs, fowl, horses, cows, goats, sheep, cetaceans, etc. In some embodiments, the subject is human.

In some examples, to induce oncolysis, kill cells, inhibit growth, inhibit metastases, decrease tumor size, or otherwise reverse or reduce the malignant phenotype of tumor cells, using the methods and compositions of the present disclosure, a cancer cell or a tumor is contacted with a therapeutically effective dose of an exemplary oncolytic virus as described herein or a pharmaceutical composition containing the same. In certain embodiments, an effective amount of an oncolytic virus as described herein or a pharmaceutical composition thereof, can include an amount sufficient to induce phagocytosis of a cancer cell, or the inhibition or reduction in the growth or size of a cancer cell. Reducing the growth of a cancer cell is manifested, for example, by cell death or reduced growth rate of a tumor comprising the cell or a prolonged survival of a subject containing the cancer cell.

Provided herein are methods for treating a subject having a cancer or a tumor comprising administering, to the subject, an effective amount of a modified virus, as described herein. An effective amount in such method includes an amount that reduces growth rate or spread of the cancer or that prolongs survival in the subject. This disclosure provides a method of treatment of cancer, activating an anti-tumor immune response, or reducing the growth of a tumor, which method comprises administering, to the tumor, an effective amount of an oncolytic virus as described herein. In certain embodiments, an effective amount of a modified virus, or a pharmaceutical composition thereof, includes an amount sufficient to induce the slowing, inhibition or reduction in the growth or size of a tumor and includes the eradication of the tumor. Reducing the growth of a tumor is manifested, for example, by reduced growth rate or a prolonged survival of a subject containing the tumor. In certain embodiments, an effective amount of a modified virus, or a pharmaceutical composition thereof, includes an amount sufficient to activate an anti-tumor response. In some embodiments, activating an anti-tumor response includes activating macrophages. In certain embodiments, an effective amount of a modified virus, or a pharmaceutical composition thereof, includes an amount sufficient to reduce incidence of tumor growth. In some embodiments, reducing incidence of tumor growth includes suppression of metastasis, prevention of primary tumor growth, suppression of existing tumor growth, or any combination thereof.

Provided herein are methods for monitoring the pharmacokinetics following administration of a therapeutically effective amount of modified oncolytic viruses according to the present disclosure, such as oncolytic vaccinia virus or a pharmaceutical composition containing the vaccinia virus, as described herein. An exemplary method for monitoring the pharmacokinetics comprises the following steps: (i) administering to the subject a therapeutically effective amount of an oncolytic virus or a pharmaceutical composition comprising the same, alone or in combination with a further therapy; (ii) collecting biological samples from the subject at one or more time points selected from about 15 minutes, about 30 minutes, about 45 mins, about 60 mins, about 75 mins, about 90 mins, about 120 mins, about 180 mins, and about 240 mins, about 5 hours, about 6 hours, about 7 hours, about 8 hours, about 9 hours, about 10 hours, about 12 hours, about 15 hours, about 24 hours, about 36 hours, about 48 hours, about 72 hours, about 4 days, about 5 days, about 6 days, about 7 days, about 8 days, about 9 days, about 10 days, about 11 days, about 12 days, about 13 days, about 14 days, about 15 days, about 1 month, to about 2 months after the administration in step (i); and (iii) detecting the quantity of the viral genome (or a reporter gene inserted within the viral genome, such as luciferase) in the biological samples collected at the above mentioned time points. In some instances, viral genome copies/mL is highest in the sample collected at the 15 mins time point and further the sample collected at the 240 mins time point does not contain a detectable quantity of the viral genome. Therefore, in some instances, a viral peak is observed at about 15 mins following administration and the majority of the virus is cleared from the subject's system after about 240 mins (or 4 hours). In some instances, a first viral peak is observed after about 15 mins following administration and a second viral peak is observed in the biological samples collected in the subsequent time points, e.g., at about 30 mins, about 45 mins, about 60 mins, or about 90 mins. The biological sample is, in exemplary embodiments, blood, and the quantity of viral genome/mL is determined by quantitative PCR or other appropriate techniques. In some examples, a first viral peak is observed after about 15 mins following administration and a second viral peak is observed after about 30 mins, about 1 hour, about 2 hours, about 3 hours, about 4 hours, about 5 hours, about 6 hours, about 7 hours, about 8 hours, about 9 hours, about 10 hours, about 12 hours, about 15 hours, about 24 hours, about 36 hours, about 48 hours, about 72 hours, about 4 days, about 5 days, about 6 days, about 7 days, about 8 days, about 9 days, about 10 days, about 11 days, about 12 days, about 13 days, about 14 days, about 15 days, about 1 month, to about 2 months following administration of an oncolytic virus as described herein.

In some instances, tumor-selective replication of an oncolytic virus is measured through use of a reporter gene, such as a luciferase gene. In some embodiments, the luciferase gene is inserted into the genome of a virus, and a tumor cell is infected with the virus. Bioluminescence in infected tumor cells is measured to monitor tumor-selective replication. Some examples show an increase in luciferase reporter bioluminescence in an oncolytic virus of this disclosure, compared to that in an otherwise identical oncolytic virus that does not contain the modifications in the oncolytic virus.

### Dosage

In some embodiments, the amount of an oncolytic virus described herein administered to a subject is between about 10³ and 10¹² infectious viral particles or plaque forming units (PFU), or between about 10⁵ and 10¹⁰ PFU, or between about 10⁵ and 10⁸ PFU, or between about 10⁸ and 10¹⁰ PFU. In some embodiments, the amount of an oncolytic virus of this disclosure administered to a subject is between about 10³ and 10¹² viral particles or plaque forming units (PFU), or between about 10⁵ and 10¹⁰ PFU, or between about 10⁵ and 10⁸ PFU, or between about 10⁸ and 10¹⁰ PFU. In some embodiments, an oncolytic virus of this disclosure is administered at a dose that comprises about 10³ PFU/dose to about 10⁴ PFU/dose, about 10⁴ PFU/dose to about 10⁵ PFU/dose, about 10⁵ PFU/dose to about 10⁶ PFU/dose, about 10⁷ PFU/dose to about 10⁸ PFU/dose, about 10⁹ PFU/dose to about 10¹⁰ PFU/dose, about 10¹⁰ PFU/dose to about 10¹¹ PFU/dose, about 10¹¹ PFU/dose to about 10¹² PFU/dose, about 10¹² PFU/dose to about 10¹³ PFU/dose, about 10¹³ PFU/dose to about 10¹⁴ PFU/dose, or about 10¹⁴ PFU/dose to about 10¹⁵ PFU/dose. In some embodiments, an oncolytic virus of this disclosure is administered at a dose that comprises about 2×10³ PFU/dose, 3×10³ PFU/dose, 4×10³ PFU/dose, 5×10³ PFU/dose, 6×10³ PFU/dose, 7×10³ PFU/dose, 8×10³ PFU/dose, 9×10³ PFU/dose, about 10⁴ PFU/dose, about 2×10⁴ PFU/dose, about 3×10⁴ PFU/dose, about 4×10⁴ PFU/dose, about 5×10⁴ PFU/dose, about 6×10⁴ PFU/dose, about 7×10⁴ PFU/dose, about 8×10⁴ PFU/dose, about 9×10⁴ PFU/dose, about 10⁵ PFU/dose, 2×10⁵ PFU/dose, 3×10⁵ PFU/dose, 4×10⁵ PFU/dose, 5×10⁵ PFU/dose, 6×10⁵ PFU/dose, 7×10⁵ PFU/dose, 8×10⁵ PFU/dose, 9×10⁵ PFU/dose, about 10⁶ PFU/dose, about 2×10⁶ PFU/dose, about 3×10⁶ PFU/dose, about 4×10⁶ PFU/dose, about 5×10⁶ PFU/dose, about 6×10⁶ PFU/dose, about 7×10⁶ PFU/dose, about 8×10⁶ PFU/dose, about 9×10⁶ PFU/dose, about 10⁷ PFU/dose, about 2×10⁷ PFU/dose, about 3×10⁷ PFU/dose, about 4×10⁷ PFU/dose, about 5×10⁷ PFU/dose, about 6×10⁷ PFU/dose, about 7×10⁷ PFU/dose, about 8×10⁷ PFU/dose, about 9×10⁷ PFU/dose, about 10⁸ PFU/dose, about 2×10⁸ PFU/dose, about 3×10⁸ PFU/dose, about 4×10⁸ PFU/dose, about 5×10⁸ PFU/dose, about 6×10⁸ PFU/dose, about 7×10⁸ PFU/dose, about 8×10⁸ PFU/dose, about 9×10⁸ PFU/dose, about 10⁹ PFU/dose, about 2×10⁹ PFU/dose, about 3×10⁹ PFU/dose, about 4×10⁹ PFU/dose, about 5×10⁹ PFU/dose, about 6×10⁹ PFU/dose, about 7×10⁹ PFU/dose, about 8×10⁹ PFU/dose, about 9×10⁹ PFU/dose, about 10¹⁰ PFU/dose, about 2×10¹⁰ PFU/dose, about 3×10¹⁰ PFU/dose, about 4×10¹⁰ PFU/dose, about 5×10¹⁰ PFU/dose, about 6×10¹⁰ PFU/dose, about 7×10¹⁰ PFU/dose, about 8×10¹⁰ PFU/dose, about 9×10¹⁰ PFU/dose, about 10¹⁰ PFU/dose, about 2×10¹⁰ PFU/dose, about 3×10¹⁰ PFU/dose, about 4×10¹⁰ PFU/dose, about 5×10¹⁰ PFU/dose, about 6×10¹⁰ PFU/dose, about 7×10¹⁰ PFU/dose, about 8×10¹⁰ PFU/dose, about 9×10¹⁰ PFU/dose, about 10¹¹ PFU/dose, about 2×10¹¹ PFU/dose, about 3×10¹¹ PFU/dose, about 4×10¹¹ PFU/dose, about 5×10¹¹ PFU/dose, about 6×10¹¹ PFU/dose, about 7×10¹¹ PFU/dose, about 8×10¹¹ PFU/dose, about 9×10¹¹ PFU/dose, or about 10¹² PFU/dose, about 10¹² PFU/dose to about 10¹³ PFU/dose, about 10¹³ PFU/dose to about 10¹⁴ PFU/dose, or about 10¹⁴ PFU/dose to about 10¹⁵ PFU/dose. In some embodiments, an oncolytic virus of this disclosure is administered at a dose that comprises 5×10⁹ PFU/dose. In some embodiments, an oncolytic virus of this disclosure is administered at a dose that comprises up to 5×10⁹ PFU/dose.

In some embodiments, an oncolytic virus of this disclosure is administered at a dose that comprises about 10³ viral particles/dose to about 10⁴ viral particles/dose, about 10⁴ viral particles/dose to about 10⁵ viral particles/dose, about 10⁵ viral particles/dose to about 10⁶ viral particles/dose, about 10⁷ viral particles/dose to about 10⁸ viral particles/dose, about 10⁹ viral particles/dose to about 10¹⁰ viral particles/dose, about 10¹⁰ viral particles/dose to about 10¹¹ viral particles/dose, about 10¹¹ viral particles/dose to about 10¹² viral particles/dose, about 10¹² viral particles/dose to about 10¹³ viral particles/dose, about 10¹³ viral particles/dose to about 10¹⁴ viral particles/dose, or about 10¹⁴ viral particles/dose to about 10¹⁵ viral particles/dose.

In some embodiments, an oncolytic virus of this disclosure is administered at a dose that comprises about 10³ PFU/kg to about 10⁴ PFU/kg, about 10⁴ PFU/kg to about 10⁵ PFU/kg, about 10⁵ PFU/kg to about 10⁶ PFU/kg, about 10⁷ PFU/kg to about 10⁸ PFU/kg, about 10⁹ PFU/kg to about 10¹⁰ PFU/kg, about 10¹⁰ PFU/kg to about 10¹¹ PFU/kg, about 10¹¹ PFU/kg to about 10¹² PFU/kg, about 10¹² PFU/kg to about 10¹³ PFU/kg, about 10¹³ PFU/kg to about 10¹⁴ PFU/kg, or about 10¹⁴ PFU/kg to about 10¹⁵ PFU/kg. In some embodiments, an oncolytic virus of this disclosure is administered at a dose that comprises about 2×10³ PFU/kg, 3×10³ PFU/kg, 4×10³ PFU/kg, 5×10³ PFU/kg, 6×10³ PFU/kg, 7×10³ PFU/kg, 8×10³ PFU/kg, 9×10³ PFU/kg, about 10⁴ PFU/kg, about 2×10⁴ PFU/kg, about 3×10⁴ PFU/kg, about 4×10⁴ PFU/kg, about 5×10⁴ PFU/kg, about 6×10⁴ PFU/kg, about 7×10⁴ PFU/kg, about 8×10⁴ PFU/kg, about 9×10⁴ PFU/kg, about 10⁵ PFU/kg, 2×10⁵ PFU/kg, 3×10⁵ PFU/kg, 4×10⁵ PFU/kg, 5×10⁵ PFU/kg, 6×10⁵ PFU/kg, 7×10⁵ PFU/kg, 8×10⁵ PFU/kg, 9×10⁵ PFU/kg, about 10⁶ PFU/kg, about 2×10⁶ PFU/kg, about 3×10⁶ PFU/kg, about 4×10⁶ PFU/kg, about 5×10⁶ PFU/kg, about 6×10⁶ PFU/kg, about 7×10⁶ PFU/kg, about 8×10⁶ PFU/kg, about 9×10⁶ PFU/kg, about 10⁷ PFU/kg, about 2×10⁷ PFU/kg, about 3×10⁷ PFU/kg, about 4×10⁷ PFU/kg, about 5×10⁷ PFU/kg, about 6×10⁷ PFU/kg, about 7×10⁷ PFU/kg, about 8×10⁷ PFU/kg, about 9×10⁷ PFU/kg, about 10⁸ PFU/kg, about 2×10⁸ PFU/kg, about 3×10⁸ PFU/kg, about 4×10⁸ PFU/kg, about 5×10⁸ PFU/kg, about 6×10⁸ PFU/kg, about 7×10⁸ PFU/kg, about 8×10⁸ PFU/kg, about 9×10⁸ PFU/kg, about 10⁹ PFU/kg, about 2×10⁹ PFU/kg, about 3×10⁹ PFU/kg, about 4×10⁹ PFU/kg, about 5×10⁹ PFU/kg, about 6×10⁹ PFU/kg, about 7×10⁹ PFU/kg, about 8×10⁹ PFU/kg, about 9×10⁹ PFU/kg, about 10¹⁰ PFU/kg, about 2×10¹⁰ PFU/kg, about 3×10¹⁰ PFU/kg, about 4×10¹⁰ PFU/kg, about 5×10¹⁰ PFU/kg, about 6×10¹⁰ PFU/kg, about 7×10¹⁰ PFU/kg, about 8×10¹⁰ PFU/kg, about 9×10¹⁰ PFU/kg, about 10¹⁰ PFU/kg, about 2×10¹⁰ PFU/kg, about 3×10¹⁰ PFU/kg, about 4×10¹⁰ PFU/kg, about 5×10¹⁰ PFU/kg, about 6×10¹⁰ PFU/kg, about 7×10¹⁰ PFU/kg, about 8×10¹⁰ PFU/kg, about 9×10¹⁰ PFU/kg, about 10¹¹ PFU/kg, about 2×10¹¹ PFU/kg, about 3×10¹¹ PFU/kg, about 4×10¹¹ PFU/kg, about 5×10¹¹ PFU/kg, about 6×10¹¹ PFU/kg, about 7×10¹¹ PFU/kg, about 8×10¹¹ PFU/kg, about 9×10¹¹ PFU/kg, or about 10¹² PFU/kg, about 10¹² PFU/kg to about 10¹³ PFU/kg, about 10¹³ PFU/kg to about 10¹⁴ PFU/kg, or about 10¹⁴ PFU/kg to about 10¹⁵ PFU/kg. In some embodiments, an oncolytic virus of this disclosure is administered at a dose that comprises 5×10⁹ PFU/kg. In some embodiments, an oncolytic virus of this disclosure is administered at a dose that comprises up to 5×10⁹ PFU/kg.

In some embodiments, an oncolytic virus of this disclosure is administered at a dose that comprises about 10³ viral particles/kg to about 10⁴ viral particles/kg, about 10⁴ viral particles/kg to about 10⁵ viral particles/kg, about 10⁵ viral particles/kg to about 10⁶ viral particles/kg, about 10⁷ viral particles/kg to about 10⁸ viral particles/kg, about 10⁹ viral particles/kg to about 10¹⁰ viral particles/kg, about 10¹⁰ viral particles/kg to about 10¹¹ viral particles/kg, about 10¹¹ viral particles/kg to about 10¹² viral particles/kg, about 10¹² viral particles/kg to about 10¹³ viral particles/kg, about 10¹³ viral particles/kg to about 10¹⁴ viral particles/kg, or about 10¹⁴ viral particles/kg to about 10¹⁵ viral particles/kg.

A liquid dosage form of an oncolytic virus as described herein comprises, in certain embodiments, a viral dose of about 10³ PFU/mL to about 10⁴ PFU/mL, about 10⁴ PFU/mL to about 10⁵ PFU/mL, about 10⁵ PFU/mL to about 10⁶ PFU/mL, about 10⁷ PFU/mL to about 10⁸ PFU/mL, about 10⁹ PFU/mL to about 10¹⁰ PFU/mL, about 10¹⁰ PFU/mL to about 10¹¹ PFU/mL, about 10¹¹ PFU/mL to about 10¹² PFU/mL, about 10¹² PFU/mL to about 10¹³ PFU/mL, about 10¹³ PFU/mL to about 10¹⁴ PFU/mL, or about 10¹⁴ PFU/mL to about 10¹⁵ PFU/mL. In some embodiments, an oncolytic virus of this disclosure is administered at a dose that comprises about 2×10³ PFU/mL, 3×10³ PFU/mL, 4×10³ PFU/mL, 5×10³ PFU/mL, 6×10³ PFU/mL, 7×10³ PFU/mL, 8×10³ PFU/mL, 9×10³ PFU/mL, about 10⁴ PFU/mL, about 2×10⁴ PFU/mL, about 3×10⁴ PFU/mL, about 4×10⁴ PFU/mL, about 5×10⁴ PFU/mL, about 6×10⁴ PFU/mL, about 7×10⁴ PFU/mL, about 8×10⁴ PFU/mL, about 9×10⁴ PFU/mL, about 10⁵ PFU/mL, 2×10⁵ PFU/mL, 3×10⁵ PFU/mL, 4×10⁵ PFU/mL, 5×10⁵ PFU/mL, 6×10⁵ PFU/mL, 7×10⁵ PFU/mL, 8×10⁵ PFU/mL, 9×10⁵ PFU/mL, about 10⁶ PFU/mL, about 2×10⁶ PFU/mL, about 3×10⁶ PFU/mL, about 4×10⁶ PFU/mL, about 5×10⁶ PFU/mL, about 6×10⁶ PFU/mL, about 7×10⁶ PFU/mL, about 8×10⁶ PFU/mL, about 9×10⁶ PFU/mL, about 10⁷ PFU/mL, about 2×10⁷ PFU/mL, about 3×10⁷ PFU/mL, about 4×10⁷ PFU/mL, about 5×10⁷ PFU/mL, about 6×10⁷ PFU/mL, about 7×10⁷ PFU/mL, about 8×10⁷ PFU/mL, about 9×10⁷ PFU/mL, about 10⁸ PFU/mL, about 2×10⁸ PFU/mL, about 3×10⁸ PFU/mL, about 4×10⁸ PFU/mL, about 5×10⁸ PFU/mL, about 6×10⁸ PFU/mL, about 7×10⁸ PFU/mL, about 8×10⁸ PFU/mL, about 9×10⁸ PFU/mL, about 10⁹ PFU/mL, about 2×10⁹ PFU/mL, about 3×10⁹ PFU/mL, about 4×10⁹ PFU/mL, about 5×10⁹ PFU/mL, about 6×10⁹ PFU/mL, about 7×10⁹ PFU/mL, about 8×10⁹ PFU/mL, about 9×10⁹ PFU/mL, about 10¹⁰ PFU/mL, about 2×10¹⁰ PFU/mL, about 3×10¹⁰ PFU/mL, about 4×10¹⁰ PFU/mL, about 5×10¹⁰ PFU/mL, about 6×10¹⁰ PFU/mL, about 7×10¹⁰ PFU/mL, about 8×10¹⁰ PFU/mL, about 9×10¹⁰ PFU/mL, about 10¹⁰ PFU/mL, about 2×10¹⁰ PFU/mL, about 3×10¹⁰ PFU/mL, about 4×10¹⁰ PFU/mL, about 5×10¹⁰ PFU/mL, about 6×10¹⁰ PFU/mL, about 7×10¹⁰ PFU/mL, about 8×10¹⁰ PFU/mL, about 9×10¹⁰ PFU/mL, about 10¹¹ PFU/mL, about 2×10¹¹ PFU/mL, about 3×10¹¹ PFU/mL, about 4×10¹¹ PFU/mL, about 5×10¹¹ PFU/mL, about 6×10¹¹ PFU/mL, about 7×10¹¹ PFU/mL, about 8×10¹¹ PFU/mL, about 9×10¹¹ PFU/mL, or about 10¹² PFU/mL, about 10¹² PFU/mL to about 10¹³ PFU/mL, about 10¹³ PFU/mL to about 10¹⁴ PFU/mL, or about 10¹⁴ PFU/mL to about 10¹⁵ PFU/mL. In some embodiments, an oncolytic virus of this disclosure is administered at a dose that comprises 5×10⁹ PFU/mL. In some embodiments, an oncolytic virus of this disclosure is administered at a dose that comprises up to 5×10⁹ PFU/mL.

In some instances, where the oncolytic virus is administered by an injection, the dosage comprises about 10³ viral particles per injection, 10⁴ viral particles per injection, 10⁵ viral particles per injection, 10⁶ viral particles per injection, 10⁷ viral particles per injection, 10⁸ viral particles per injection, 10⁹ viral particles per injection, 10¹⁰ viral particles per injection, 10¹¹ viral particles per injection, 10¹² viral particles per injection, 2×10¹² viral particles per injection, 10¹³ viral particles per injection, 10¹⁴ viral particles per injection, or 10¹⁵ viral particles per injection. In further instances, where the oncolytic virus is administered by an injection, the dosage comprises about 10³ infectious viral particles per injection, 10⁴ infectious viral particles per injection, 10⁵ infectious viral particles per injection, 10⁶ infectious viral particles per injection, 10⁷ infectious viral particles per injection, 10⁸ infectious viral particles per injection, 10⁹ infectious viral particles per injection, 10¹⁰ infectious viral particles per injection, 10¹¹ infectious viral particles per injection, 10¹² infectious viral particles per injection, 2×10¹² infectious viral particles per injection, 10¹³ infectious viral particles per injection, 10¹⁴ infectious viral particles per injection, or 10¹⁵ infectious viral particles per injection. In certain embodiments, the virus is administered in an amount sufficient to induce oncolysis in at least about 20% of cells in a tumor, in at least about 30% of cells in a tumor, in at least about 40% of cells in a tumor, in at least about 50% of cells in a tumor, in at least about 60% of cells in a tumor, in at least about 70% of cells in a tumor, in at least about 80% of cells in a tumor, or in at least about 90% of cells in a tumor. In certain embodiments, a single dose of virus refers to the amount administered to a subject or a tumor over a 1, 2, 5, 10, 15, 20 or 24 hour period. In certain embodiments, the dose is spread over time or by separate injection. In certain embodiments, multiple doses (e.g., 2, 3, 4, 5, 6 or more doses) of the vaccinia virus is administered to the subject, for example, where a second treatment occurs within 1, 2, 3, 4, 5, 6, 7 days or weeks of a first treatment. In certain embodiments, multiple doses of the oncolytic virus is administered to the subject over a period of 1, 2, 3, 4, 5, 6, 7 or more days or weeks. In certain embodiments, the oncolytic virus or the pharmaceutical composition as described herein is administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. The frequency of administration of the oncolytic virus or the pharmaceutical composition as described herein is, in certain instances, once daily, twice daily, once every week, once every three weeks, once every four weeks (or once a month), once every 8 weeks (or once every 2 months), once every 12 weeks (or once every 3 months), or once every 24 weeks (once every 6 months). In some embodiments of the methods disclosed herein, the oncolytic virus or the pharmaceutical composition is administered, independently, in an initial dose for a first period of time, an intermediate dose for a second period of time, and a high dose for a third period of time. In some embodiments, the initial dose is lower than the intermediate dose and the intermediate dose is lower than the high dose. In some embodiments, the first, second, and third periods of time are, independently, about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer.

An exemplary method for the delivery of an oncolytic virus as described herein or a pharmaceutical composition comprising the same, to cancer or tumor cells is via intravenous administration, e.g., intravenous, via infusion, parenteral, intravenous, intradermal, intramuscular, transdermal, rectal, intraurethral, intravaginal, intranasal, intrathecal, or intraperitoneal. However, alternate methods of administration are also used, e.g., via intratumoral injection. The routes of administration vary with the location and nature of the tumor. In certain embodiments, the route of administration is intradental, transdermal, parenteral, intraperitoneal, intravenous, intramuscular, intranasal, subcutaneous, regional (e.g., in the proximity of a tumor, particularly with the vasculature or adjacent vasculature of a tumor), percutaneous, intrathecal, intratracheal, intraperitoneal, intraarterial, intravesical, intratumoral, inhalation, perfusion, by lavage or orally. An injectable dose of the oncolytic virus is administered as a bolus injection or as a slow infusion. In certain embodiments, the oncolytic virus is administered to the patient from a source implanted in the patient. In certain embodiments, administration of the oncolytic virus occurs by continuous infusion over a selected period of time. In some instances, an oncolytic vaccinia virus as described herein, or a pharmaceutical composition containing the same is administered at a therapeutically effective dose by infusion over a period of about 15 mins, about 30 mins, about 45 mins, about 50 mins, about 55 mins, about 60 minutes, about 75 mins, about 90 mins, about 100 mins, or about 120 mins or longer. The oncolytic virus or the pharmaceutical composition of the present disclosure is administered as a liquid dosage, wherein the total volume of administration is about 1 mL to about 5 mL, about 5 mL to 10 mL, about 15 mL to about 20 mL, about 25 mL to about 30 mL, about 30 mL to about 50 mL, about 50 mL to about 100 mL, about 100 mL to 150 mL, about 150 mL to about 200 mL, about 200 mL to about 250 mL, about 250 mL to about 300 mL, about 300 mL to about 350 mL, about 350 mL to about 400 mL, about 400 mL to about 450 mL, about 450 mL to 500 mL, about 500 mL to 750 mL, or about 750 mL to 1000 mL.

### Formulations

Pharmaceutical compositions containing a modified virus, such as an oncolytic virus, as described herein, are prepared as solutions, dispersions in glycerol, liquid polyethylene glycols, and any combinations thereof in oils, in solid dosage forms, as inhalable dosage forms, as intranasal dosage forms, as liposomal formulations, dosage forms comprising nanoparticles, dosage forms comprising microparticles, polymeric dosage forms, or any combinations thereof. In some embodiments, a pharmaceutical composition as described herein comprises a stabilizer and a buffer. In some embodiments, a pharmaceutical composition as described herein can comprise a solubilizer, such as sterile water or Tris-buffer. In some embodiments, a pharmaceutical composition as described herein can comprise an excipient. Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, or a coloring agent.

In certain embodiments, a buffering agent includes phosphate buffered saline (PBS), Dulbecco's PBS (DPBS), TRIS-buffered saline (TBS), Hank's balanced salt solution (HBSS), Earle's balanced salt solution (EBSS), standard saline citrate (SSC), HEPES-buffered saline (HBS), or Gey's balanced salt solution.

In certain embodiments, a pharmaceutical composition of this disclosure comprises an effective amount of a modified virus, disclosed herein, combined with a pharmaceutically acceptable carrier. “Pharmaceutically acceptable,” as used herein, includes any carrier which does not interfere with the effectiveness of the biological activity of the active ingredients and/or that is not toxic to the patient to whom it is administered. Non-limiting examples of suitable pharmaceutical carriers include phosphate buffered saline solutions, water, emulsions, such as oil/water emulsions, various types of wetting agents and sterile solutions. Additional non-limiting examples of pharmaceutically compatible carriers include gels, bioadsorbable matrix materials, implantation elements containing the oncolytic virus or any other suitable vehicle, delivery or dispensing means or material. Such carriers are formulated by conventional methods and are administered to the subject at an effective amount.

### Methods of Production

The oncolytic viruses of this disclosure are produced by methods known to one of skill in the art. In certain embodiments, the oncolytic virus is propagated in suitable host cells, e.g., HeLa cells, 293 cells, or Vero cells, isolated from host cells and stored in conditions that promote stability and integrity of the virus, such that loss of infectivity over time is minimized. In certain exemplary methods, the oncolytic viruses are propagated in host cells using cell stacks, roller bottles, or perfusion bioreactors. In some examples, downstream methods for purification of the oncolytic viruses comprise filtration (e.g., depth filtration, tangential flow filtration, or a combination thereof), ultracentrifugation, or chromatographic capture. The oncolytic virus is stored, e.g., by freezing or drying, such as by lyophilization. In certain embodiments, prior to administration, the stored modified oncolytic virus is reconstituted (if dried for storage) and diluted in a pharmaceutically acceptable carrier for administration.

Some embodiments provide that the oncolytic virus as described herein, exhibit a higher titer in HeLa cells and 293 cells compared to an otherwise identical virus that does not comprise the modifications in the oncolytic virus. In certain instances, a higher titer in HeLa cells and 293 cells is seen in modified oncolytic virus.

### Kits

In embodiments, this disclosure provides for a kit for administering an oncolytic virus as described herein. In certain embodiments, a kit of this disclosure includes an oncolytic virus or a pharmaceutical composition comprising an oncolytic virus as described above. In certain embodiments, a kit of this disclosure further includes one or more components such as instructions for use, devices and additional reagents, and components, such as tubes, containers, and syringes for performing the methods disclosed above. In certain embodiments, a kit of this disclosure further includes one or more agents, e.g., at least one of an anti-cancer agent, an immunomodulatory agent, or any combinations thereof, that is administered in combination with a modified virus.

In certain embodiments, a kit of this disclosure comprises one or more containers containing a modified virus, disclosed herein. For example, and not by way of limitation, a kit of this disclosure comprises one or more containers that contain an oncolytic virus of this disclosure.

In certain embodiments, a kit of this disclosure includes instructions for use, a device for administering the oncolytic virus to a subject, or a device for administering an additional agent or compound to a subject. For example, and not by way of limitation, the instructions include a description of the oncolytic virus and, optionally, other components included in the kit, and methods for administration, including methods for determining the proper state of the subject, the proper dosage amount and the proper administration method for administering the modified virus. Instructions also optionally include guidance for monitoring the subject over duration of the treatment time.

In certain embodiments, a kit of this disclosure includes a device for administering the oncolytic virus to a subject. Any of a variety of devices known in the art for administering medications and pharmaceutical compositions is included in the kits provided herein. For example, and not by way of limitation, such devices include, a hypodermic needle, an intravenous needle, a catheter, a needle-less injection device, an inhaler, and a liquid dispenser, such as an eyedropper. In certain embodiments, an oncolytic virus to be delivered systemically, for example, by intravenous injection, an intra-tumoral injection, an intraperitoneal injection, is included in a kit with a hypodermic needle and syringe.

While preferred embodiments of this disclosure have been shown and described herein, it will be obvious to those skilled in the art that such embodiments are provided by way of example only. Numerous variations, changes, and substitutions will now occur to those skilled in the art without departing from this disclosure. It should be understood that various alternatives to the embodiments of this disclosure described herein are employed in practicing the disclosure. It is intended that the following claims define the scope of the disclosure and that methods and structures within the scope of these claims and their equivalents be covered thereby.

### Exemplary Embodiments

Provided herein are compositions, wherein the composition comprises: an oncolytic virus comprising an exogenous nucleic acid comprising a sequence encoding for a soluble SIRP-alpha polypeptide or functional fragment thereof. Further provided herein are compositions, wherein the polypeptide comprises a CD47 binding region. Further provided herein are compositions, wherein the SIRP-alpha polypeptide comprises an IgV domain. Further provided herein are compositions, wherein the polypeptide binds CD47 with a higher binding constant than a native SIRP-alpha. Further provided herein are compositions, wherein the SIRP-alpha polypeptide lacks a transmembrane domain. Further provided herein are compositions, wherein the SIRP-alpha polypeptide is from a murine SIRP-alpha, a human SIRP-alpha, or any combination thereof. Further provided herein are compositions, wherein the sequence encoding for the SIRP-alpha polypeptide comprises a nucleic acid sequence having at least 85%, 90%, 95%, or 99% sequence identity to SEQ ID NO: 2 or SEQ ID NO: 6. Further provided herein are compositions, wherein the sequence encoding for the SIRP-alpha polypeptide comprises a nucleic acid sequence of SEQ ID NO: 2 or SEQ ID NO: 6. Further provided herein are compositions, wherein the SIRP-alpha polypeptide comprises an amino acid sequence having at least 85%, 90%, 95%, or 99% sequence identity to SEQ ID NO: 11 or SEQ ID NO: 15. Further provided herein are compositions, wherein the SIRP-alpha polypeptide comprises an amino acid sequence of either SEQ ID NO: 11 or SEQ ID NO: 15. Further provided herein are compositions, further comprising at least one promoter region. Further provided herein are compositions, wherein the at least one promoter region drives expression of the polypeptide. Further provided herein are compositions, wherein the at least one promoter comprises any one of P7.5, P10, P28, SSP, P135, 454, TK promoter, E/L, F7L, H5R, H1L, A1L, J3R, E4L, I1L, I5L, I7L, T7, I2L, FP4b, ATI, P11, PFL1, PH5, L4R, 28 kDa promoter, or any variation or combination thereof. Further provided herein are compositions, wherein the promoter comprises the P7.5 promoter. Further provided herein are compositions, wherein the P7.5 promoter comprises a nucleic acid sequence having at least 85%, 90%, 95%, or 99% sequence identity to SEQ ID NO: 23. Further provided herein are compositions, wherein the P7.5 promoter comprises the sequence of SEQ ID NO: 23. Further provided herein are compositions, wherein the promoter comprises the P10 promoter. Further provided herein are compositions, wherein the P10 promoter comprises a sequence having at least 85%, 90%, 95%, or 99% sequence identity to SEQ ID NO: 24. Further provided herein are compositions, wherein the P10 promoter comprises the sequence of SEQ ID NO: 24.

Provided herein are compositions, wherein the composition comprises: an oncolytic virus comprising an exogenous nucleic acid comprising a sequence encoding for an anti-CD47 antibody or fragment thereof. Further provided herein are compositions, wherein the anti-CD47 antibody comprises an immunoglobulin G (IgG) domain. Further provided herein are compositions, wherein the IgG domain comprises an Fc region. Further provided herein are compositions, wherein the anti-CD47 antibody is a humanized monoclonal antibody. Further provided herein are compositions, wherein the anti-CD47 antibody comprises amino acid sequences having at least 85%, 90%, 95%, or 99% sequence identity to SEQ ID NO: 21 and SEQ ID NO: 22. Further provided herein are compositions, wherein the anti-CD47 antibody comprises amino acid sequences as in SEQ ID NO: 21 and SEQ ID NO: 22. Further provided herein are compositions, wherein the anti-CD47 antibody comprises magrolimab. Further provided herein are compositions, wherein the Fc region comprises an IgG1 Fc. Further provided herein are compositions, wherein the Fc region comprises an IgG4 Fc. Further provided herein are compositions, further comprising at least one promoter region. Further provided herein are compositions, wherein the at least one promoter region drives expression of the polypeptide. Further provided herein are compositions, wherein the at least one promoter comprises any one of P7.5, P10, P28, SSP, P135, 454, TK promoter, E/L, F7L, H5R, H1L, A1L, J3R, E4L, I1L, I5L, I7L, T7, I2L, FP4b, ATI, P11, PFL1, PH5, L4R, 28 kDa promoter, or any variation or combination thereof. Further provided herein are compositions, wherein the promoter comprises the P7.5 promoter. Further provided herein are compositions, wherein the P7.5 promoter comprises a nucleic acid sequence having at least 85%, 90%, 95%, or 99% sequence identity to SEQ ID NO: 23. Further provided herein are compositions, wherein the P7.5 promoter comprises the sequence of SEQ ID NO: 23. Further provided herein are compositions, wherein the promoter comprises the P10 promoter. Further provided herein are compositions, wherein the P10 promoter comprises a sequence having at least 85%, 90%, 95%, or 99% sequence identity to SEQ ID NO: 24. Further provided herein are compositions, wherein the P10 promoter comprises the sequence of SEQ ID NO: 24.

Provided herein are compositions as described herein, wherein the oncolytic virus is a poxvirus, an adeno associated virus, an adenovirus, a reovirus, a lentivirus, a herpes simplex virus, a vesicular stomatitis virus, a mengovirus, a myxoma virus, Newcastle disease virus, measles virus, or polio virus. Further described herein are compositions, wherein the poxvirus is a vaccinia virus. Further described herein are compositions, wherein the vaccinia virus is a modified strain of Western Reserve Vaccinia virus (ATCC VR-1354), Vaccinia virus Ankara (ATCC VR-1508), Vaccinia virus Ankara (ATCC VR-1566), Vaccinia virus strain Wyeth (ATCC VR-1536), or Vaccinia virus Wyeth (ATCC VR-325). Further described herein are compositions, wherein the exogenous nucleic acid is inserted into the viral genome. Further described herein are compositions, wherein the oncolytic virus comprises at least one genome modification. Further described herein are compositions, wherein the at least one modification comprises a mutation or deletion of at least one gene selected from the group consisting of: Thymidine Kinase, F13L, A36R, A34R, A33R, A52R, B5R, B8R, B18R, SPI-1, SPI-2, B15R, VGF, E3L, K3L, A41L, K7R, or N1L, a functional fragment thereof, or any combinations thereof. Further described herein are compositions, comprising a mutation or deletion of the TK gene. Further described herein are compositions, further comprising a mutation or deletion of the B8R gene.

Provided herein are compositions, wherein the composition comprises: an oncolytic virus, wherein the oncolytic virus comprises: an insertion at a TK gene locus comprising, in 5′ to 3′ order: a promoter region, wherein the promoter is P7.5; and a region encoding a soluble SIRP-alpha polypeptide or functional fragment thereof.

Provided herein are compositions, wherein the composition comprises: an oncolytic virus, wherein the oncolytic virus comprises: an insertion at a TK gene locus comprising, in 5′ to 3′ order: a promoter region, wherein the promoter is P10; a region encoding a soluble SIRP-alpha polypeptide or functional fragment thereof.

Provided herein are compositions, wherein the composition comprises: an oncolytic virus, wherein the oncolytic virus comprises: an insertion at a TK gene locus comprising, in 5′ to 3′ order: a promoter region, wherein the promoter is P7.5; and a region encoding an anti-CD47 antibody or fragment thereof.

Provided herein are compositions, wherein the composition comprises: an oncolytic virus, wherein the oncolytic virus comprises: an insertion at a TK gene locus comprising, in 5′ to 3′ order: a promoter region, wherein the promoter is P10; a region encoding an anti-CD47 antibody or fragment thereof.

Provided herein are compositions, wherein the pharmaceutical composition comprises: a composition as described herein; and a pharmaceutically acceptable excipient. Further provided herein are compositions, wherein pharmaceutical the composition is in a liquid dosage form. Further provided herein are compositions, wherein the pharmaceutically acceptable excipient is a buffered saline. Further provided herein are compositions, wherein the buffered saline is phosphate buffered saline (PBS), Dulbecco's PBS (DPBS), TRIS-buffered saline (TBS), Hank's balanced salt solution (HBSS), Earle's balanced salt solution (EBSS), standard saline citrate (SSC), HEPES-buffered saline (HBS), or Gey's balanced salt solution. Further provided herein are compositions, wherein the pharmaceutical composition further comprises a liposome or nanoparticle. Further provided herein are compositions, wherein the nucleic acid or vector is associated with the liposome or nanoparticle.

Provided herein are methods for treatment of cancer comprising administering to a subject having cancer a pharmaceutical composition as described herein in an amount sufficient for treatment of a cancer. Further provided herein are methods, wherein the cancer is a solid tumor, a leukemia, or a lymphoma. Further provided herein are methods, wherein the cancer comprises melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, myeloproliferative neoplasms, or sarcoma. Further provided herein are methods, wherein the administering comprises an intravenous administration. Further provided herein are methods, wherein the administering comprises an intratumoral administration. Further provided herein are methods, wherein the administering comprises a systemic administration. Further provided herein are methods, wherein the systemic administration comprises oral administration, parenteral administration, intranasal administration, sublingual administration, rectal administration, transdermal administration, or any combinations thereof.

Provided herein are methods for activating an anti-tumor immune response, comprising administering to a subject having a cancer a pharmaceutical composition as described herein. Further provided herein are methods, wherein the cancer is a solid tumor, a leukemia, or a lymphoma. Further provided herein are methods, wherein the cancer comprises melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, myeloproliferative neoplasms, or sarcoma. Further provided herein are methods, wherein the administering step is an intravenous administration. Further provided herein are methods, wherein the administering step is an intratumoral administration. Further provided herein are methods, wherein the administering step is a systemic administration. Further provided herein are methods, wherein the systemic administration comprises oral administration, parenteral administration, intranasal administration, sublingual administration, rectal administration, transdermal administration, or any combinations thereof.

Provided herein are methods for reduction of incidence of tumor cell growth, comprising: administering to tumor cells a pharmaceutical composition as described herein in an effective amount sufficient for reduction of incidence of tumor cell growth. Further provided herein are methods, wherein the tumor cells are from a solid, a leukemia, or a lymphoma. Further provided herein are methods, wherein the tumor cells are from a melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, myeloproliferative neoplasms, or sarcoma. Further provided herein are methods, wherein the administering step is an intravenous administration. Further provided herein are methods, wherein the administering step is an intratumoral administration.

### EXAMPLES

The examples below further illustrate the described embodiments without limiting the scope of this disclosure.

### Example 1: Construction of Murine SIRP-Alpha Expression System

A vaccinia virus, Western Reserve strain, is modified by replacing the gene encoding thymidine kinase (VACV094, J2R) with a nucleic acid encoding a soluble domain of murine SIRP-alpha and a fluorescent reporter. A plasmid transfer vector is generated comprising, in order, with no gaps, a 5′ recombination directing sequence (SEQ ID NO: 30), an SbfI cloning site (CCTGCAGG), a P7.5 promoter (SEQ ID NO: 23), a Spel cloning site (ACTAGT), soluble domain of murine SIRP-alpha (SEQ ID NO: 2), a sacI cloning site (GAGCTC), a loxP sequence (SEQ ID NO: 31), a spacer sequence A (SEQ ID NO: 32), viral 454 promoter (SEQ ID NO: 26), fluorescent reporter protein (SEQ ID NO: 33), a PacI cloning site (TTAATTAA), a short spacer B (SEQ ID NO: 34), a loxP sequence (SEQ ID NO: 31), and a 3′ recombination directing sequence (SEQ ID NO: 35). Following recombination and treatment with cre recombinase, the viral genome comprises the incorporated sequence of SEQ ID NO: 36. Selected sequences are shown in Table 5. A schematic representation of the promoter and transgene inserted at a TK locus is shown in FIG. 2.

TABLE 5
soluble domain of SIRP-alpha recombination sequences.

| SEQ | | |
|---|---|---|
| ID NO: | Description | Nucleic acid sequence |
| 30 | 5′ recombination-directing | ATTTACGGATTCACCAATAAAAATAAACTAGAG |
| | sequence | AAACTTAGTACTAATAAGGAACTAGAATCGTAT |
| | | AGTTCTAGCCCTCTTCAAGAACCCATTAGGTTAA |
| | | ATGATTTTCTGGGACTATTGGAATGTGTTAAAAA |
| | | GAATATTCCTCTAACAGATATTCCGACAAAGGA |
| | | TTGATTACTATAAATGGAGAATGTTCCTAATGTA |
| | | TACTTTAATCCTGTGTTTATAGAGCCCACGTTTA |
| | | AACATTCTTTATTAAGTGTTTATAAACACAGATT |
| | | AATAGTTTTATTTGAAGTATTCGTTGTATTCATTC |
| | | TAATATATGTATTTTTTAGATCTGAATTAAATAT |
| | | GTTCTTCATGCCTAAACGAAAAATACCCGATCCT |
| | | ATTGATAGATTACGACGTGCTAATCTAGCGTGTG |
| | | AAGACGATAAATTAATGATCTATGGATTACCAT |
| | | GGATGACAACTCAAACATCTGCGTTATCAATAA |
| | | ATAGTAAACCGATAGTGTATAAAGATTGTGCAA |
| | | AGCTTTTGCGATCAATAAATGGATCACAACCAG |
| | | TATCTCTTAACGATGTTCTTCGCAGATGATGATT |
| | | CATTTTTTAAGTATTTGGCTAGTCAAGATGATGA |
| | | ATCTTCATTATCTGATATATTGCAAATCACTCAA |
| | | TATCTAGACTTTCTGTTATTATTATTGATCCAATC |
| | | AAAAAATAAATTAGAAGCCGTGGGTCATTGTTA |
| | | TGAATCTCTTTCAGAGGAATACAGACAATTGAC |
| | | AAAATTCACAGACTTTCAAGATTTTAAAAAACT |
| | | GTTTAACAAGGTCCCTATTGTTACAGATGGAAG |
| | | GGTCAAACTTAATAAAGGATATTTGTTCGACTTT |
| | | GTGATTAGTTTGATGCGATTCAAAAAAGAATCCT |
| | | CTCTAGCTACCACCGCAATAGATCCTGTTAGATA |
| | | CATAGATCCTCGTCGCAATATCGCATTTTCTAAC |
| | | GTGATGGATATATTAAAGTCGAATAAAGTGAAC |
| | | AATAATTAATTCTTTATTGTCATC |
| 31 | LoxP sequence | ATAACTTCGTATAGCATACATTATACGAAGTTAT |
| 32 | Spacer sequence A | AATTCTGCAGAT |
| 33 | Fluorescent reporter protein | ATGCGTAAAGGCGAAGAGCTGTTCACTGGTGTC |
| | | GTCCCTATTCTGGTGGAACTGGATGGTGATGTCA |
| | | ACGGTCATAAGTTTTCCGTGCGTGGCGAGGGTG |
| | | AAGGTGACGCAACTAATGGTAAACTGACGCTGA |
| | | AGTTCATCTGTACTACTGGTAAACTGCCGGTCCC |
| | | TTGGCCGACTCTGGTAACGACGCTGACTTATGGT |
| | | GTTCAGTGCTTTGCTCGTTATCCGGACCATATGA |
| | | AGCAGCATGACTTCTTCAAGTCCGCCATGCCGG |
| | | AAGGCTATGTGCAGGAACGCACGATTTCCTTTA |
| | | AGGATGACGGCACGTACAAAACGCGTGCGGAAG |
| | | TGAAATTTGAAGGCGATACCCTGGTAAACCGCA |
| | | TTGAGCTGAAAGGCATTGACTTTAAAGAAGACG |
| | | GCAATATCCTGGGCCATAAGCTGGAATACAATT |
| | | TTAACAGCCACAATGTTTACATCACCGCCGATAA |
| | | ACAAAAAAATGGCATTAAAGCGAATTTTAAAAT |
| | | TCGCCACAACGTGGAGGATGGCAGCGTGCAGCT |
| | | GGCTGATCACTACCAGCAAAACACTCCAATCGG |
| | | TGATGGTCCTGTTCTGCTGCCAGACAATCACTAT |
| | | CTGAGCACGCAAAGCGTTCTGTCTAAAGATCCG |
| | | AACGAGAAACGCGATCATATGGTTCTGCTGGAG |
| | | TTCGTAACCGCAGCGGGCATCACGCATGGTATG |
| | | GATGAACTGTACAAATAA |
| 34 | Spacer sequence B | AAGGCCGCTCGA |
| 35 | Homologous recombination- | TATTATATTTTTTATCTAAAAAACTAAAAATAAA |
| | directing sequence, 3′ | CATTGATTAAATTTTAATATAATACTTAAAAATG |
| | | GATGTTGTGTCGTTAGATAAACCGTTTATGTATT |
| | | TTGAGGAAATTGATAATGAGTTAGATTACGAAC |
| | | CAGAAAGTGCAAATGAGGTCGCAAAAAAACTGC |
| | | CGTATCAAGGACAGTTAAAACTATTACTAGGAG |
| | | AATTATTTTTTCTTAGTAAGTTACAGCGACACGG |
| | | TATATTAGATGGTGCCACCGTAGTGTATATAGGA |
| | | TCTGCTCCCGGTACACATATACGTTATTTGAGAG |
| | | ATCATTTCTATAATTTAGGAGTGATCATCAAATG |
| | | GATGCTAATTGACGGCCGCCATCATGATCCTATT |
| | | TTAAATGGATTGCGTGATGTGACTCTAGTGACTC |
| | | GGTTCGTTGATGAGGAATATCTACGATCCATCAA |
| | | AAAACAACTGCATCCTTCTAAGATTATTTTAATT |
| | | TCTGATGTGAGATCCAAACGAGGAGGAAATGAA |
| | | CCTAGTACGGCGGATTTACTAAGTAATTACGCTC |
| | | TACAAAATGTCATGATTAGTATTTTAAACCCCGT |
| | | GGCGTCTAGTCTTAAATGGAGATGCCCGTTTCCA |
| | | GATCAATGGATCAAGGACTTTTATATCCCACACG |
| | | GTAATAAAATGTTACAACCTTTTGCTCCTTCATA |
| | | TTCAGCTGAAATGAGATTATTAAGTATTTATACC |
| | | GGTGAGAACATGAGACTGACTCGAGTTACCAAA |
| | | TCAGACGCTGTAAATTATGAAAAAAAGATGTAC |
| | | TACCTTAATAAGATCGTCCGTAACAAAGTAGTTG |
| | | TTAACTTTGATTATCCTAATCAGGAATATGACTA |
| | | TTTTCACATGTACTTTATGCTGAGGACCGTGTAC |
| | | TGCAATAAAACATTTCCTACTACTAAAGCAAAG |
| | | GTACTATTTCTACAACAATCTATATTTCGTTTCTT |
| | | AAATATTCCAACAACATCAACTGAAAAAGTTAG |
| | | TCATGAACCAATACAACG |
| 36 | Final sequence in viral genome | ATTTACGGATTCACCAATAAAAATAAACTAGAG |
| | (non-native DNA BOLD; | AAACTTAGTACTAATAAGGAACTAGAATCGTAT |
| | transgene open reading frames | AGTTCTAGCCCTCTTCAAGAACCCATTAGGTTAA |
| | UNDERLINED) | ATGATTTTCTGGGACTATTGGAATGTGTTAAAAA |
| | | GAATATTCCTCTAACAGATATTCCGACAAAGGA |
| | | TTGATTACTATAAATGGAGAATGTTCCTAATGTA |
| | | TACTTTAATCCTGTGTTTATAGAGCCCACGTTTA |
| | | AACATTCTTTATTAAGTGTTTATAAACACAGATT |
| | | AATAGTTTTATTTGAAGTATTCGTTGTATTCATTC |
| | | TAATATATGTATTTTTTAGATCTGAATTAAATAT |
| | | GTTCTTCATGCCTAAACGAAAAATACCCGATCCT |
| | | ATTGATAGATTACGACGTGCTAATCTAGCGTGTG |
| | | AAGACGATAAATTAATGATCTATGGATTACCAT |
| | | GGATGACAACTCAAACATCTGCGTTATCAATAA |
| | | ATAGTAAACCGATAGTGTATAAAGATTGTGCAA |
| | | AGCTTTTGCGATCAATAAATGGATCACAACCAG |
| | | TATCTCTTAACGATGTTCTTCGCAGATGATGATT |
| | | CATTTTTTAAGTATTTGGCTAGTCAAGATGATGA |
| | | ATCTTCATTATCTGATATATTGCAAATCACTCAA |
| | | TATCTAGACTTTCTGTTATTATTATTGATCCAAT |
| | | CAAAAAATAAATTAGAAGCCGTGGGTCATTGTTA |
| | | TGAATCTCTTTCAGAGGAATACAGACAATTGAC |
| | | AAAATTCACAGACTTTCAAGATTTTAAAAAACT |
| | | GTTTAACAAGGTCCCTATTGTTACAGATGGAAG |
| | | GGTCAAACTTAATAAAGGATATTTGTTCGACTTT |
| | | GTGATTAGTTTGATGCGATTCAAAAAAGAATCCT |
| | | CTCTAGCTACCACCGCAATAGATCCTGTTAGATA |
| | | CATAGATCCTCGTCGCAATATCGCATTTTCTAAC |
| | | GTGATGGATATATTAAAGTCGAATAAAGTGAAC |
| | | AATAATTAATTCTTTATTGTCATCCCTGCAGGTC |
| | | ACTAATTCCAAACCCACCCGCTTTTTATAGTA |
| | | AGTTTTTCACCCATAAATAATAAATACAATAA |
| | | TTAATTTCTCGTAAAAGTAGAAAATATATTCT |
| | | AATTTATTGCACGGTAAGGAAGTAGATCATAA |
| | | ACTAGTATGGAGCCCGCCGGCCCCGCCCCCG |
| | | GCAGGCTGGGCCCCCTGCTGCTGTGCCTGCT |
| | | GCTGAGCGCCAGCTGCTTCTGCACCGGCGCC |
| | | ACCGGCAAGGAGCTGAAGGTGACCCAGCCCG |
| | | AGAAGAGCGTGAGCGTGGCCGCCGGCGACAG |
| | | CACCGTGCTGAACTGCACCCTGACCAGCCTG |
| | | CTGCCCGTGGGCCCCATCAGGTGGTACAGGG |
| | | GCGTGGGCCCCAGCAGGCTGCTGATCTACAG |
| | | CTTCGCCGGCGAGTACGTGCCCAGGATCAGG |
| | | AACGTGAGCGACACCACCAAGAGGAACAACA |
| | | TGGACTTCAGCATCAGGATCAGCAACGTGAC |
| | | CCCCGCCGACGCCGGCATCTACTACTGCGTG |
| | | AAGTTCCAGAAGGGCAGCAGCGAGCCCGACA |
| | | CCGAGATCCAGAGCGGCGGCGGCACCGAGGT |
| | | GGAGCTCATAACTTCGTATAGCATACATTATA |
| | | CGAAGTTATTATTATATTTTTTATCTAAAAAACT |
| | | AAAAATAAACATTGATTAAATTTTAATATAATAC |
| | | TTAAAAATGGATGTTGTGTCGTTAGATAAACCGT |
| | | TTATGTATTTTGAGGAAATTGATAATGAGTTAGA |
| | | TTACGAACCAGAAAGTGCAAATGAGGTCGCAAA |
| | | AAAACTGCCGTATCAAGGACAGTTAAAACTATT |
| | | ACTAGGAGAATTATTTTTTCTTAGTAAGTTACAG |
| | | CGACACGGTATATTAGATGGTGCCACCGTAGTG |
| | | TATATAGGATCTGCTCCCGGTACACATATACGTT |
| | | ATTTGAGAGATCATTTCTATAATTTAGGAGTGAT |
| | | CATCAAATGGATGCTAATTGACGGCCGCCATCA |
| | | TGATCCTATTTTAAATGGATTGCGTGATGTGACT |
| | | CTAGTGACTCGGTTCGTTGATGAGGAATATCTAC |
| | | GATCCATCAAAAAACAACTGCATCCTTCTAAGA |
| | | TTATTTTAATTTCTGATGTGAGATCCAAACGAGG |
| | | AGGAAATGAACCTAGTACGGCGGATTTACTAAG |
| | | TAATTACGCTCTACAAAATGTCATGATTAGTATT |
| | | TTAAACCCCGTGGCGTCTAGTCTTAAATGGAGAT |
| | | GCCCGTTTCCAGATCAATGGATCAAGGACTTTTA |
| | | TATCCCACACGGTAATAAAATGTTACAACCTTTT |
| | | GCTCCTTCATATTCAGCTGAAATGAGATTATTAA |
| | | GTATTTATACCGGTGAGAACATGAGACTGACTC |
| | | GAGTTACCAAATCAGACGCTGTAAATTATGAAA |
| | | AAAAGATGTACTACCTTAATAAGATCGTCCGTA |
| | | ACAAAGTAGTTGTTAACTTTGATTATCCTAATCA |
| | | GGAATATGACTATTTTCACATGTACTTTATGCTG |
| | | AGGACCGTGTACTGCAATAAAACATTTCCTACTA |
| | | CTAAAGCAAAGGTACTATTTCTACAACAATCTAT |
| | | ATTTCGTTTCTTAAATATTCCAACAACATCAACT |
| | | GAAAAAGTTAGTCATGAACCAATACAACG |

### Example 2: Construction of Human SIRP-Alpha Expression System

A vaccinia virus, Western Reserve strain, is modified by replacing the gene encoding thymidine kinase (VACV094, J2R) with a nucleic acids encoding a soluble domain of human SIRP-alpha and a fluorescent reporter. A plasmid transfer vector is generated comprising, in order, with no gaps, a 5′ recombination directing sequence (SEQ ID NO: 30), an SbfI cloning site (CCTGCAGG), a P7.5 promoter (SEQ ID NO: 23), Spel cloning site (ACTAGT), open reading frame encoding a domain of human SIRP-alpha (SEQ ID NO: 6), a SacI cloning site (GAGCTC), a loxP sequence (SEQ ID NO: 31), a spacer sequence A (SEQ ID NO: 32), promoter 454 (SEQ ID NO: 26), fluorescent reporter protein (SEQ ID NO: 33), a PacI cloning site (TTAATTAA), spacer sequence B (SEQ ID NO: 34), a loxP sequence (SEQ ID NO: 31), and a 3′ recombination directing sequence (SEQ ID NO: 35). Following recombination and treatment with cre recombinase, the viral genome comprises the incorporated sequence of SEQ ID NO: 37. Sequences are shown in Table 6.

TABLE 6
Human IL-12 and a domain of human SIRP-alpha recombination sequences.

| SEQ ID | | |
|---|---|---|
| NO: | Description | Nucleic acid sequence |
| 37 | Final sequence in viral | ATTTACGGATTCACCAATAAAAATAAACTAGAGA |
| | genome (non-native DNA | AACTTAGTACTAATAAGGAACTAGAATCGTATA |
| | BOLD; transgene open | GTTCTAGCCCTCTTCAAGAACCCATTAGGTTAA |
| | reading frames | ATGATTTTCTGGGACTATTGGAATGTGTTAAAA |
| | UNDERLINED) | AGAATATTCCTCTAACAGATATTCCGACAAAGG |
| | | ATTGATTACTATAAATGGAGAATGTTCCTAATG |
| | | TATACTTTAATCCTGTGTTTATAGAGCCCACGT |
| | | TTAAACATTCTTTATTAAGTGTTTATAAACACA |
| | | GATTAATAGTTTTATTTGAAGTATTCGTTGTAT |
| | | TCATTCTAATATATGTATTTTTTAGATCTGAAT |
| | | TAAATATGTTCTTCATGCCTAAACGAAAAATAC |
| | | CCGATCCTATTGATAGATTACGACGTGCTAATC |
| | | TAGCGTGTGAAGACGATAAATTAATGATCTATG |
| | | GATTACCATGGATGACAACTCAAACATCTGCGT |
| | | TATCAATAAATAGTAAACCGATAGTGTATAAAG |
| | | ATTGTGCAAAGCTTTTGCGATCAATAAATGGAT |
| | | CACAACCAGTATCTCTTAACGATGTTCTTCGCA |
| | | GATGATGATTCATTTTTTAAGTATTTGGCTAGT |
| | | CAAGATGATGAATCTTCATTATCTGATATATTG |
| | | CAAATCACTCAATATCTAGACTTTCTGTTATTA |
| | | TTATTGATCCAATCAAAAAATAAATTAGAAGCC |
| | | GTGGGTCATTGTTATGAATCTCTTTCAGAGGAA |
| | | TACAGACAATTGACAAAATTCACAGACTTTCAA |
| | | GATTTTAAAAAACTGTTTAACAAGGTCCCTATT |
| | | GTTACAGATGGAAGGGTCAAACTTAATAAAGGA |
| | | TATTTGTTCGACTTTGTGATTAGTTTGATGCGA |
| | | TTCAAAAAAGAATCCTCTCTAGCTACCACCGCA |
| | | ATAGATCCTGTTAGATACATAGATCCTCGTCGC |
| | | AATATCGCATTTTCTAACGTGATGGATATATTA |
| | | AAGTCGAATAAAGTGAACAATAATTAATTCTTT |
| | | ATTGTCATCCCTGCAGGTCACTAATTCCAAACC |
| | | CACCCGCTTTTTATAGTAAGTTTTTCACCCATA |
| | | AATAATAAATACAATAATTAATTTCTCGTAAAA |
| | | GTAGAAAATATATTCTAATTTATTGCACGGTAA |
| | | GGAAGTAGATCATAAACTAGTATGTGCCACCAG |
| | | CAGCTGGTGATCAGCTGGTTCAGCCTGGTGTTC |
| | | CTGGCCAGCCCCCTGGTGGCCATCTGGGAGCTG |
| | | AAGAAGGACGTGTACGTGGTGGAGCTGGACTGG |
| | | TACCCCGACGCCCCCGGCGAGATGGTGGTGCTG |
| | | ACCTGCGACACCCCCGAGGAGGACGGCATCACC |
| | | TGGACCCTGGACCAGAGCAGCGAGGTGCTGGGC |
| | | AGCGGCAAGACCCTGACCATCCAGGTGAAGGAG |
| | | TTCGGCGACGCCGGCCAGTACACCTGCCACAAG |
| | | GGCGGCGAGGTGCTGAGCCACAGCCTGCTGCTG |
| | | CTGCACAAGAAGGAGGACGGCATCTGGAGCACC |
| | | GACATCCTGAAGGACCAGAAGGAGCCCAAGAAC |
| | | AAGACCTTCCTGAGGTGCGAGGCCAAGAACTAC |
| | | AGCGGCAGGTTCACCTGCTGGTGGCTGACCACC |
| | | ATCAGCACCGACCTGACCTTCAGCGTGAAGAGC |
| | | AGCAGGGGCAGCAGCGACCCCCAGGGCGTGACC |
| | | TGCGGCGCCGCCACCCTGAGCGCCGAGAGGGTG |
| | | AGGGGCGACAACAAGGAGTACGAGTACAGCGTG |
| | | GAGTGCCAGGAGGACAGCGCCTGCCCCGCCGCC |
| | | GAGGAGAGCCTGCCCATCGAGGTGATGGTGGAC |
| | | GCCGTGCACAAGCTGAAGTACGAGAACTACACC |
| | | AGCAGCTTCTTCATCAGGGACATCATCAAGCCC |
| | | GACCCCCCCAAGAACCTGCAGCTGAAGCCCCTG |
| | | AAGAACAGCAGGCAGGTGGAGGTGAGCTGGGAG |
| | | TACCCCGACACCTGGAGCACCCCCCACAGCTAC |
| | | TTCAGCCTGACCTTCTGCGTGCAGGTGCAGGGC |
| | | AAGAGCAAGAGGGAGAAGAAGGACAGGGTGTTC |
| | | ACCGACAAGACCAGCGCCACCGTGATCTGCAGG |
| | | AAGAACGCCAGCATCAGCGTGAGGGCCCAGGAC |
| | | AGGTACTACAGCAGCAGCTGGAGCGAGTGGGCC |
| | | AGCGTGCCCTGCAGCGGCGGCGGCGGCAGCGGC |
| | | GGCGGCGGCAGCGGCGGCGGCGGCAGCGGCGGC |
| | | GGCGGCAGCGCCAGCTGGCCCCCCGGCAGCGCC |
| | | AGCCAGCCCCCCCCCAGCCCCGCCGCCGCCACC |
| | | GGCCTGCACCCCGCCGCCAGGCCCGTGAGCCTG |
| | | CAGTGCAGGCTGAGCATGTGCCCCGCCAGGAGC |
| | | CTGCTGCTGGTGGCCACCCTGGTGCTGCTGGAC |
| | | CACCTGAGCCTGGCCAGGAACCTGCCCGTGGCC |
| | | ACCCCCGACCCCGGCATGTTCCCCTGCCTGCAC |
| | | CACAGCCAGAACCTGCTGAGGGCCGTGAGCAAC |
| | | ATGCTGCAGAAGGCCAGGCAGACCCTGGAGTTC |
| | | TACCCCTGCACCAGCGAGGAGATCGACCACGAG |
| | | GACATCACCAAGGACAAGACCAGCACCGTGGAG |
| | | GCCTGCCTGCCCCTGGAGCTGACCAAGAACGAG |
| | | AGCTGCCTGAACAGCAGGGAGACCAGCTTCATC |
| | | ACCAACGGCAGCTGCCTGGCCAGCAGGAAGACC |
| | | AGCTTCATGATGGCCCTGTGCCTGAGCAGCATC |
| | | TACGAGGACCTGAAGATGTACCAGGTGGAGTTC |
| | | AAGACCATGAACGCCAAGCTGCTGATGGACCCC |
| | | AAGAGGCAGATCTTCCTGGACCAGAACATGCTG |
| | | GCCGTGATCGACGAGCTGATGCAGGCCCTGAAC |
| | | TTCAACAGCGAGACCGTGCCCCAGAAGAGCAGC |
| | | CTGGAGGAGCCCGACTTCTACAAGACCAAGATC |
| | | AAGCTGTGCATCCTGCTGCACGCCTTCAGGATC |
| | | AGGGCCGTGACCATCGACAGGGTGATGAGCTAC |
| | | CTGAACGCCAGCGAGCTCATAACTTCGTATAGC |
| | | ATACATTATACGAAGTTATTATTATATTTTTTA |
| | | TCTAAAAAACTAAAAATAAACATTGATTAAATT |
| | | TTAATATAATACTTAAAAATGGATGTTGTGTCG |
| | | TTAGATAAACCGTTTATGTATTTTGAGGAAATT |
| | | GATAATGAGTTAGATTACGAACCAGAAAGTGCA |
| | | AATGAGGTCGCAAAAAAACTGCCGTATCAAGGA |
| | | CAGTTAAAACTATTACTAGGAGAATTATTTTTT |
| | | CTTAGTAAGTTACAGCGACACGGTATATTAGAT |
| | | GGTGCCACCGTAGTGTATATAGGATCTGCTCCC |
| | | GGTACACATATACGTTATTTGAGAGATCATTTC |
| | | TATAATTTAGGAGTGATCATCAAATGGATGCTA |
| | | ATTGACGGCCGCCATCATGATCCTATTTTAAAT |
| | | GGATTGCGTGATGTGACTCTAGTGACTCGGTTC |
| | | GTTGATGAGGAATATCTACGATCCATCAAAAAA |
| | | CAACTGCATCCTTCTAAGATTATTTTAATTTCT |
| | | GATGTGAGATCCAAACGAGGAGGAAATGAACCT |
| | | AGTACGGCGGATTTACTAAGTAATTACGCTCTA |
| | | CAAAATGTCATGATTAGTATTTTAAACCCCGTG |
| | | GCGTCTAGTCTTAAATGGAGATGCCCGTTTCCA |
| | | GATCAATGGATCAAGGACTTTTATATCCCACAC |
| | | GGTAATAAAATGTTACAACCTTTTGCTCCTTCA |
| | | TATTCAGCTGAAATGAGATTATTAAGTATTTAT |
| | | ACCGGTGAGAACATGAGACTGACTCGAGTTACC |
| | | AAATCAGACGCTGTAAATTATGAAAAAAAGATG |
| | | TACTACCTTAATAAGATCGTCCGTAACAAAGTA |
| | | GTTGTTAACTTTGATTATCCTAATCAGGAATAT |
| | | GACTATTTTCACATGTACTTTATGCTGAGGACC |
| | | GTGTACTGCAATAAAACATTTCCTACTACTAAA |
| | | GCAAAGGTACTATTTCTACAACAATCTATATTT |
| | | CGTTTCTTAAATATTCCAACAACATCAACTGAA |
| | | AAAGTTAGTCATGAACCAATACAACG |

### Example 3: Deletion of B8R Gene (IFNg Binding Protein)

Interferon gamma (IFNg) plays a role in anticancer immunity by promoting activity of various immune cells. The vaccinia IFNg binding protein, expressed by the B8R gene, acts as a secreted decoy receptor, removing extracellular IFNg. Virus as described in Example 1 or 2 is further modified to remove the B8R gene.

A transfer vector is generated comprising, in order, a reporter vector 5′ recombination directing sequence (SEQ ID NO: 38), a spacer sequence C (SEQ ID NO: 39), a 454 promoter (SEQ ID NO: 26), a fluorescent reporter protein (SEQ ID NO: 40), a reporter vector 3′ recombination directing sequence (SEQ ID NO: 41). Components of the vector replace the B8R open reading frame with a nucleic acid expressing fluorescent reporter gene for plaque selection purposes. Selected plaques are then treated with a second transfer vector to remove the promoter and fluorescent reporter gene, comprising the reporter vector 5′ recombination directing sequence (SEQ ID NO: 38) and reporter vector 3′ recombination directing sequence (SEQ ID NO: 41). The final sequence of the modified B8R locus is as in SEQ ID NO: 42. The original location of the B8R open reading frame is designated by paired brackets [ ]. Sequences are shown in Table 7.

TABLE 7
B8R removal vector sequences

| SEQ | | |
|---|---|---|
| ID NO: | Description | Nucleic acid sequence |
| 38 | Reporter vector, 5′ | GTACGTACTATGCCAAATGAATCACGTGTTAAA |
| | recombination-directing | CAATGGGTGATGGATACACTTAATGGTATAATG |
| | sequence | ATGAATGAACGCGATGTTTCTGTAAGCGTTGGC |
| | | ACCGGAATACTATTCATGGAAATGTTTTTCGATT |
| | | ACAATAAAAATAGTATCAACAATCAACTAATGT |
| | | ATGATATAATTAATAGCGTATCTATAATTCTAGC |
| | | TAATGAGAGATATAGAAGCGCTTTTAACGACGA |
| | | TGGTATATACATCCGTAGAAATATGATTAACAA |
| | | GTTGTACGGATACGCATCTCTAACTACTATTGGC |
| | | ACGATCGCTGGAGGTGTTTGTTATTATCTGTTGA |
| | | TGCATCTAGTTAGTTTGTATAAATAATTATTTCA |
| | | ATATACTAGTTAAAATTTTAAGATTTTAAATGTA |
| | | TAAAAAACTAATAACGTTTTTATTTGTAATAGGT |
| | | GCATTAGCATCCTATTCGAATAATGAGTACACTC |
| | | CGTTTAATAAACTGAGTGTAAAACTCTATATAGA |
| | | TGGAGTAGATAATATAGAAAATTCATATACTGA |
| | | TGATAATAATGAATTGGTGTTAAATTTTAAAGAG |
| | | TACACAATTTCTATTATTACAGAGTCATGCGACG |
| | | TCGGATTTGATTCCATAGATATAGATGTTATAAA |
| | | CGACTATAAAATTATTGATATGTATACCATTGAC |
| | | TCGTCTACTATTCAACGCAGAGGTCACACGTGTA |
| | | GAATATCTACCAAATTATCATGCCATTATGATAA |
| | | GTACCCTTATATTCACAAATATGATGGTGATGAG |
| | | CGACAATATTCTATTACTGCAGAGGGAAAATGC |
| | | TATAAAGGAATAAAATATGAAATAAGTATGATC |
| | | AACGATGATACTCTATTGAGAAAACATACTCTTA |
| | | AAATTGGATCTACTTATATATTTGATCGTCATGG |
| | | ACATAGTAATACATATTATTCAAAATATGATTTT |
| | | TAAAAATTTAAAATATATTATCACTTCAGTGACA |
| | | GTAGTCAAATAACAAACAACACC |
| 39 | Spacer sequence C | CGAAGTTATAATTCTGCAGA |
| 40 | fluorescent reporter protein | ATGCGTAAAGGCGAAGAGCTGTTCACTGGTGTC |
| | | GTCCCTATTCTGGTGGAACTGGATGGTGATGTCA |
| | | ACGGTCATAAGTTTTCCGTGCGTGGCGAGGGTG |
| | | AAGGTGACGCAACTAATGGTAAACTGACGCTGA |
| | | AGTTCATCTGTACTACTGGTAAACTGCCGGTCCC |
| | | TTGGCCGACTCTGGTAACGACGCTGACTTATGGT |
| | | GTTCAGTGCTTTGCTCGTTATCCGGACCATATGA |
| | | AGCAGCATGACTTCTTCAAGTCCGCCATGCCGG |
| | | AAGGCTATGTGCAGGAACGCACGATTTCCTTTA |
| | | AGGATGACGGCACGTACAAAACGCGTGCGGAAG |
| | | TGAAATTTGAAGGCGATACCCTGGTAAACCGCA |
| | | TTGAGCTGAAAGGCATTGACTTTAAAGAAGACG |
| | | GCAATATCCTGGGCCATAAGCTGGAATACAATT |
| | | TTAACAGCCACAATGTTTACATCACCGCCGATAA |
| | | ACAAAAAAATGGCATTAAAGCGAATTTTAAAAT |
| | | TCGCCACAACGTGGAGGATGGCAGCGTGCAGCT |
| | | GGCTGATCACTACCAGCAAAACACTCCAATCGG |
| | | TGATGGTCCTGTTCTGCTGCCAGACAATCACTAT |
| | | CTGAGCACGCAAAGCGTTCTGTCTAAAGATCCG |
| | | AACGAGAAACGCGATCATATGGTTCTGCTGGAG |
| | | TTCGTAACCGCAGCGGGCATCACGCATGGTATG |
| | | GATGAACTGTACAAATAG |
| 41 | Reporter vector, 3′ | CTAATTTTTATTAATGATACAAAAACGAAATAA |
| | recombination-directing | AACTGCATATTATACACTGGTTAACGCCCTTATA |
| | sequence | GGCTCTAACCATTTTCAAGATGAGGTCCCTGATT |
| | | ATAGTCCTTCTGTTCCCCTCTATCATCTACTCCAT |
| | | GTCTATTAGACAATGTGAGAAGACTGAAGAGGA |
| | | AACATGGGGATTGAAAATAGGGTTGTGTATAAT |
| | | TGCCAAAGATTTCTATCCCGAAAGAACTGATTGC |
| | | AGTGTTCATCTCCCAACTGCAAGTGAAGGATTG |
| | | ATAACTGAAGGCAATGGATTCAGGGATATACGA |
| | | AACACCGATAAATTATAAAAAAAGCAATGTGTC |
| | | CGCTGTTTCCGTTAATAATACTATTTTCGTAACT |
| | | GGCGGATTATTCATAAATAACTCTAATAGCACG |
| | | ATCGTGGTTAACAATATGGAAAAACTTGACATTT |
| | | ATAAAGACAAACAATGGTCGATTATAGAAATGC |
| | | CTATGGCTAGGGTATATCACGGCATCGACTCGA |
| | | CATTTGGAATGTTATATTTTGCCGGAGGTCTATC |
| | | CGTTACCGAACAATATGGTAATTTAGAGAAAAA |
| | | CAACGAGATATCTTGTTACAATCCTAGAACGAA |
| | | TAAGTGGTTTGATATTTCATATACTATTTATAAG |
| | | ATATCCATATCATCATTGTGTAAACTAAATAACG |
| | | TCTTCTATGTATTTAGTAAGGACATTGGATATGT |
| | | GGAAAAGTATGATGGTGCATGGAAGTTAGTACA |
| | | TGATCGTCTCCCCGCTATAAAGGCATTATCAACT |
| | | TCTCCTTATTGATTGAAAATGAAAATATAAATAG |
| | | TTTTTATGTATAGCAGTATTACCCTATAGTTTTAT |
| | | TGCTTACTACTAACATGGATACAGATG |
| 42 | The sequence of the modified | GTACGTACTATGCCAAATGAATCACGTGTTAAA |
| | (deleted) B8R locus is | CAATGGGTGATGGATACACTTAATGGTATAATG |
| | (original location of B8R open | ATGAATGAACGCGATGTTTCTGTAAGCGTTGGC |
| | reading frame is designated by | ACCGGAATACTATTCATGGAAATGTTTTTCGATT |
| | paired brackets []) | ACAATAAAAATAGTATCAACAATCAACTAATGT |
| | | ATGATATAATTAATAGCGTATCTATAATTCTAGC |
| | | TAATGAGAGATATAGAAGCGCTTTTAACGACGA |
| | | TGGTATATACATCCGTAGAAATATGATTAACAA |
| | | GTTGTACGGATACGCATCTCTAACTACTATTGGC |
| | | ACGATCGCTGGAGGTGTTTGTTATTATCTGTTGA |
| | | TGCATCTAGTTAGTTTGTATAAATAATTATTTCA |
| | | ATATACTAGTTAAAATTTTAAGATTTTAAATGTA |
| | | TAAAAAACTAATAACGTTTTTATTTGTAATAGGT |
| | | GCATTAGCATCCTATTCGAATAATGAGTACACTC |
| | | CGTTTAATAAACTGAGTGTAAAACTCTATATAGA |
| | | TGGAGTAGATAATATAGAAAATTCATATACTGA |
| | | TGATAATAATGAATTGGTGTTAAATTTTAAAGAG |
| | | TACACAATTTCTATTATTACAGAGTCATGCGACG |
| | | TCGGATTTGATTCCATAGATATAGATGTTATAAA |
| | | CGACTATAAAATTATTGATATGTATACCATTGAC |
| | | TCGTCTACTATTCAACGCAGAGGTCACACGTGTA |
| | | GAATATCTACCAAATTATCATGCCATTATGATAA |
| | | GTACCCTTATATTCACAAATATGATGGTGATGAG |
| | | CGACAATATTCTATTACTGCAGAGGGAAAATGC |
| | | TATAAAGGAATAAAATATGAAATAAGTATGATC |
| | | AACGATGATACTCTATTGAGAAAACATACTCTTA |
| | | AAATTGGATCTACTTATATATTTGATCGTCATGG |
| | | ACATAGTAATACATATTATTCAAAATATGATTTT |
| | | TAAAAATTTAAAATATATTATCACTTCAGTGACA |
| | | GTAGTCAAATAACAAACAACACC[]CTAATTTTTA |
| | | TTAATGATACAAAAACGAAATAAAACTGCATAT |
| | | TATACACTGGTTAACGCCCTTATAGGCTCTAACC |
| | | ATTTTCAAGATGAGGTCCCTGATTATAGTCCTTC |
| | | TGTTCCCCTCTATCATCTACTCCATGTCTATTAGA |
| | | CAATGTGAGAAGACTGAAGAGGAAACATGGGG |
| | | ATTGAAAATAGGGTTGTGTATAATTGCCAAAGA |
| | | TTTCTATCCCGAAAGAACTGATTGCAGTGTTCAT |
| | | CTCCCAACTGCAAGTGAAGGATTGATAACTGAA |
| | | GGCAATGGATTCAGGGATATACGAAACACCGAT |
| | | AAATTATAAAAAAAGCAATGTGTCCGCTGTTTCC |
| | | GTTAATAATACTATTTTCGTAACTGGCGGATTAT |
| | | TCATAAATAACTCTAATAGCACGATCGTGGTTAA |
| | | CAATATGGAAAAACTTGACATTTATAAAGACAA |
| | | ACAATGGTCGATTATAGAAATGCCTATGGCTAG |
| | | GGTATATCACGGCATCGACTCGACATTTGGAAT |
| | | GTTATATTTTGCCGGAGGTCTATCCGTTACCGAA |
| | | CAATATGGTAATTTAGAGAAAAACAACGAGATA |
| | | TCTTGTTACAATCCTAGAACGAATAAGTGGTTTG |
| | | ATATTTCATATACTATTTATAAGATATCCATATC |
| | | ATCATTGTGTAAACTAAATAACGTCTTCTATGTA |
| | | TTTAGTAAGGACATTGGATATGTGGAAAAGTAT |
| | | GATGGTGCATGGAAGTTAGTACATGATCGTCTCC |
| | | CCGCTATAAAGGCATTATCAACTTCTCCTTATTG |
| | | ATTGAAAATGAAAATATAAATAGTTTTTATGTAT |
| | | AGCAGTATTACCCTATAGTTTTATTGCTTACTAC |
| | | TAACATGGATACAGATG |

The foregoing description and accompanying drawings set forth a number of representative embodiments at the present time. Various modifications, additions, and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A composition, wherein the composition comprises:
an oncolytic virus comprising an exogenous nucleic acid comprising a sequence encoding for a soluble SIRP-alpha polypeptide or functional fragment thereof.

2. The composition of claim 1, wherein the SIRP-alpha polypeptide comprises a CD47 binding region.

3. The composition of claim 2, wherein the SIRP-alpha polypeptide comprises an IgV domain.

4. The composition of claim 2, wherein the SIRP-alpha polypeptide binds CD47 with a higher binding constant than a native SIRP-alpha.

5. The composition of claim 1, wherein the SIRP-alpha polypeptide lacks a transmembrane domain.

6. The composition of claim 1, wherein the SIRP-alpha polypeptide is from a murine SIRP-alpha, a human SIRP-alpha, or any combination thereof.

7. The composition of claim 6, wherein the sequence encoding for the SIRP-alpha polypeptide comprises a nucleic acid sequence having at least 85%, 90%, 95%, 99%, or 100% sequence identity to SEQ ID NO: 2 or SEQ ID NO: 6.

8. The composition of claim 6, wherein the SIRP-alpha polypeptide comprises an amino acid sequence having at least 85%, 90%, 95%, 99%, or 100% sequence identity to SEQ ID NO: 11 or SEQ ID NO: 15.

9. The composition of claim 1, further comprising at least one promoter region.

10. The composition of claim 9, wherein the at least one promoter region comprises any one of P7.5, P10, P28, SSP, P135, 454, TK, E/L, F7L, H5R, H1L, A1L, J3R, E4L, I1L, I5L, I7L, T7, I2L, FP4b, ATI, P11, PFL1, PH5, L4R, 28 kDa promoter, or any variation or combination thereof.

11. The composition of claim 10, wherein the promoter comprises the P7.5 promoter, and wherein the P7.5 promoter comprises a nucleic acid sequence having at least 85%, 90%, 95%, 99%, or 100% sequence identity to SEQ ID NO: 23.

12. The composition of claim 10, wherein the promoter comprises the P10 promoter, and wherein the P10 promoter comprises a sequence having at least 85%, 90%, 95%, 99%, or 100% sequence identity to SEQ ID NO: 24.

13. The composition of claim 1, wherein the oncolytic virus is a poxvirus, an adeno associated virus, an adenovirus, a reovirus, a lentivirus, a herpes simplex virus, a vesicular stomatitis virus, a mengovirus, a myxoma virus, a Newcastle disease virus, a measles virus, or a polio virus.

14. The composition of claim 13, wherein the poxvirus is a vaccinia virus.

15. The composition of claim 14, wherein the vaccinia virus is a modified strain of Western Reserve Vaccinia virus (ATCC VR-1354), Vaccinia virus Ankara (ATCC VR-1508), Vaccinia virus Ankara (ATCC VR-1566), Vaccinia virus strain Wyeth (ATCC VR-1536), or Vaccinia virus Wyeth (ATCC VR-325).

16. The composition of claim 1, wherein the exogenous nucleic acid is inserted into the a genome of the oncolytic virus.

17. The composition of claim 1, wherein the oncolytic virus comprises at least one genome modification.

18. The composition of claim 17, wherein the at least one genome modification comprises a mutation or deletion of at least one gene selected from the group consisting of:
Thymidine Kinase (TK), F13L, A36R, A34R, A33R, A52R, B5R, B8R, B18R, SPI-1, SPI-2, B15R, VGF, E3L, K3L, A41L, K7R, N1L, a functional fragment thereof, and any combinations thereof.

19. The composition of claim 18, wherein the at least one modification comprises a mutation or deletion of the TK gene.

20. The composition of claim 19, wherein the at least one modification further comprises a mutation or deletion of the B8R gene.

21. A composition, wherein the composition comprises:
an oncolytic virus, wherein the oncolytic virus comprises:
an insertion at a TK gene locus comprising, in 5′ to 3′ order:
a promoter region, wherein the promoter is P7.5; and
a region encoding a soluble SIRP-alpha polypeptide or functional fragment thereof.

22. A pharmaceutical composition, wherein the pharmaceutical composition comprises:
the composition of claim 1; and
a pharmaceutically acceptable excipient.

23. The pharmaceutical composition of claim 22, wherein the pharmaceutically acceptable excipient is a buffered saline.

24. The pharmaceutical composition of claim 22, wherein the pharmaceutical composition further comprises a liposome or nanoparticle.
